(19) United States
(12) Patent Application Publication
(10) Pub. No.: US 2026/0275386 A1
(43) Pub. Date: Sep. 17, 2026

(54) Compositions and Methods for Genome Editing

(71) Applicants: The Trustees of the University of Pennsylvania, Philadelphia, PA (US); The Regents of the University of California, Oakland, CA (US)

(72) Inventors: Connor A. Tsuchida, Berkeley, CA (US); Jennifer A. Doudna, Berkeley, CA (US); Carl H. June, Merion Station, PA (US); Joseph A. Fraietta, Williamstown, NJ (US)

(21) Appl. No.: 19/167,298

(22) PCT Filed: Mar. 19, 2024
(86) PCT No.: PCT/US2024/020601

§ 371 Date: Sep. 19, 2025

Related U.S. Application Data
(60) Provisional application No. 63/453,631, filed on Mar. 21, 2023.

(51) Int. Cl. C12N 15/90; C07K 14/47; C12N 9/22; C12N 15/11; C12N 15/86

(52) U.S. Cl. CPC C12N 15/907; C07K 14/4702; C12N 9/226; C12N 15/11; C12N 15/86; C12N 2310/20; C12N 2740/15043; C12N 2750/14143

(57) ABSTRACT

The present disclosure provides methods for modifying the genome of a mammalian cell, the method comprising contacting the mammalian cell in vitro with a gene editing composition and an agent that increases the level of a p53 polypeptide in the cell, thereby generating a modified cell. The present disclosure provides treatment methods, comprising generating a modified cell, and administering the modified cell to an individual in need thereof. The present disclosure provides gene editing compositions comprising a CRISPR-Cas effector polypeptide, a guide nucleic acid, and an agent that increases the level of a p53 polypeptide in a mammalian cell.

### CROSS-REFERENCE

This application claims the benefit of U.S. Provisional Patent Application No. 63/453,631 filed Mar. 21, 2023, which application is incorporated herein by reference in its entirety.

### STATEMENT REGARDING FEDERALLY SPONSORED RESEARCH

This invention was made with government support under Grant Numbers AI142817, HG009490, and HL156468 awarded by the National Institutes of Health. The government has certain rights in the invention.

### INCORPORATION BY REFERENCE OF SEQUENCE LISTING PROVIDED AS AN XML FILE

A Sequence Listing is provided herewith as a Sequence Listing XML, “BERK-484WO_SeqList.xml” created on Mar. 19, 2024 and having a size of 6,698 bytes. The contents of the Sequence Listing XML are incorporated by reference herein in their entirety.

### INTRODUCTION

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. Genome editing can be carried out using a CRISPR-Cas system comprising a CRISPR-Cas effector polypeptide and a guide nucleic acid, such as a guide RNA. However, unintended chromosomal abnormalities following on-target genome editing, such as chromosome loss, are potential concerns for genome editing.

### SUMMARY

The present disclosure provides methods for modifying the genome of a mammalian cell, the method comprising contacting the mammalian cell in vitro with a gene editing composition and an agent that increases the level of a p53 polypeptide in the cell, thereby generating a modified cell. The present disclosure provides treatment methods, comprising generating a modified cell, and administering the modified cell to an individual in need thereof. The present disclosure provides gene editing compositions comprising a CRISPR-Cas effector polypeptide, a guide nucleic acid, and an agent that increases the level of a p53 polypeptide in a mammalian cell.

### BRIEF DESCRIPTION OF THE DRAWINGS

FIG. 1A-1H depict whole and partial chromosome loss following CRISPR-Cas9 genome editing of TRAC.

FIG. 2A-2E depict target-specific chromosome loss revealed by a genome-scale CRISPR-Cas screen.

FIG. 3A-3D depict the influence of genetic and epigenetic factors on Cas9-induced chromosome loss.

FIG. 4A-4B depict persistence of Cas9-induced chromosome loss.

FIG. 5A-5D depict chromosome loss following pre-clinical CAR T cell production via homology-directed repair.

FIG. 6A-6E depict the effect of clinical CRISPR-Cas9 genome editing protocol on chromosome loss in patient T cells.

FIG. 7A-7I depict data showing that CRISPR-Cas9 genome editing results in chromosome loss regardless of guide RNA orientation or genomic position.

FIG. 8A-8F depict genome-scale breakpoints and chromosome loss revealed by CROP-seq.

FIG. 9A-9G depict the influence of genetic context and Cas9 gRNA sequence on chromosome loss.

FIG. 10A-10D depict data showing that Cas9-induced chromosome loss is associated with differential gene expression and a fitness disadvantage.

FIG. 11A-11E depict the effect of CRISPR-Cas9 homology-directed repair on chromosome loss.

FIG. 12A-12D depict reduced chromosome loss in T cells with clinical CRISPR-Cas9 genome editing protocol.

FIG. 13A-13E provide amino acid sequences of p53 polypeptides.

### DEFINITIONS

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, humans, non-human primates, ungulates, felines, canines, bovines, ovines, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an agent that increases the level of a p53 polypeptide in a mammalian cell” includes a plurality of such agents and reference to “the CRISPR-Cas effector polypeptide” includes reference to one or more CRISPR-Cas effector polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for modifying the genome of a mammalian cell, the method comprising contacting the mammalian cell in vitro with a gene editing composition and an agent that increases the level of a p53 polypeptide in the cell. The present disclosure provides gene editing compositions comprising a CRISPR-Cas effector polypeptide, a guide nucleic acid, and an agent that increases the level of a p53 polypeptide in a mammalian cell.

### Methods for Modifying the Genome of a Mammalian Cell

The present disclosure provides a method for modifying the genome of a mammalian cell in vitro. The method generally involves contacting the mammalian cell in vitro with: a) a gene-editing composition; and b) an agent that increases the level of a p53 polypeptide in the mammalian cell. The contacting generates a modified (gene edited) mammalian cell (i.e., an in vitro modified mammalian cell).

In some cases, the step of contacting the mammalian cell with an agent that increases the level of a p53 polypeptide is carried out before the step of contacting the mammalian cell with a gene-editing composition. For example, in some cases, the step of contacting the mammalian cell with an agent that increases the level of a p53 polypeptide is carried out from 1 hour to about 72 hours before the step of contacting the mammalian cell with a gene-editing composition. In some cases, the step of contacting the mammalian cell with an agent that increases the level of a p53 polypeptide is carried out from 1 hour to about 4 hours before the step of contacting the mammalian cell with a gene-editing composition. In some cases, the step of contacting the mammalian cell with an agent that increases the level of a p53 polypeptide is carried out from 4 hours to about 8 hours before the step of contacting the mammalian cell with a gene-editing composition. In some cases, the step of contacting the mammalian cell with an agent that increases the level of a p53 polypeptide is carried out from 8 hours to about 12 hours before the step of contacting the mammalian cell with a gene-editing composition. In some cases, the step of contacting the mammalian cell with an agent that increases the level of a p53 polypeptide is carried out from 12 hours to about 48 hours before the step of contacting the mammalian cell with a gene-editing composition. In some cases, the step of contacting the mammalian cell with an agent that increases the level of a p53 polypeptide is carried out from 12 hours to about 24 hours before the step of contacting the mammalian cell with a gene-editing composition. In some cases, the step of contacting the mammalian cell with an agent that increases the level of a p53 polypeptide is carried out from 12 hours to about 36 hours before the step of contacting the mammalian cell with a gene-editing composition. In some cases, the step of contacting the mammalian cell with an agent that increases the level of a p53 polypeptide is carried out from 24 hours to about 36 hours before the step of contacting the mammalian cell with a gene-editing composition. In some cases, the step of contacting the mammalian cell with an agent that increases the level of a p53 polypeptide is carried out from 24 hours to about 48 hours before the step of contacting the mammalian cell with a gene-editing composition. In some cases, the step of contacting the mammalian cell with an agent that increases the level of a p53 polypeptide is carried out from 48 hours to about 72 hours before the step of contacting the mammalian cell with a gene-editing composition.

In some cases, the step of contacting the mammalian cell with an agent that increases the level of a p53 polypeptide is carried out substantially simultaneously with the step of contacting the mammalian cell with a gene-editing composition. For example, in some cases, the step of contacting the mammalian cell with an agent that increases the level of a p53 polypeptide is carried out within about 1 hour of the step of contacting the mammalian cell with a gene-editing composition. In some cases, the step of contacting the mammalian cell with an agent that increases the level of a p53 polypeptide is carried out within from 1 minute to 1 hour of the step of contacting the mammalian cell with a gene-editing composition. In some cases, the step of contacting the mammalian cell with an agent that increases the level of a p53 polypeptide is carried out within from 1 minute to 45 minutes of the step of contacting the mammalian cell with a gene-editing composition. In some cases, the step of contacting the mammalian cell with an agent that increases the level of a p53 polypeptide is carried out within from 1 minute to 30 minutes of the step of contacting the mammalian cell with a gene-editing composition. In some cases, the step of contacting the mammalian cell with an agent that increases the level of a p53 polypeptide is carried out within from 1 minute to 15 minutes of the step of contacting the mammalian cell with a gene-editing composition. In some cases, the step of contacting the mammalian cell with a gene editing composition is carried out at the same time as the step of contacting the mammalian cell with an agent that increases the level of p53 polypeptide in the cell.

In some cases, a method of the present disclosure results in fewer chromosomal abnormalities in the mammalian cell, compared to the number of chromosomal abnormalities that occur using the same method but without the step of contacting the mammalian cell with an agent that increases the level of a p53 polypeptide. Chromosomal abnormalities that can be decreased using a method of the present disclosure include, e.g., translocations, large deletions, loss of heterozygosity, and chromothripsis. In some cases, a method of the present disclosure results in at least 10%, at least 15%, at least 20%, at least 25%, at least 30%, at least 35%, at least 40%, at least 45%, or at least 50%, fewer chromosomal abnormalities in the mammalian cell, compared to the number of chromosomal abnormalities that occur using the same method but without the step of contacting the mammalian cell with an agent that increases the level of a p53 polypeptide.

The genome modification is mediated by the CRISPR-Cas effector polypeptide, which is guided to a target nucleic acid in the genome by the guide nucleic acid. Contacting a target nucleic acid in the genome with a CRISPR-Cas effector polypeptide complexed with a guide nucleic acid generates site-specific double strand breaks (DSBs) or single strand breaks within double-stranded DNA (dsDNA) target nucleic acids, which are repaired either by non-homologous end joining (NHEJ) or homology-directed recombination (HDR). In some cases, contacting a target nucleic acid in the genome with a CRISPR-Cas effector polypeptide complexed with a guide nucleic acid generates a DSB.

In some cases, a method of the present disclosure further comprises stimulating and activating the mammalian cell. For example, where the mammalian cell is a CD3⁺ T cell, the method can include contacting the CD3⁺ T cell with one or more cytokines, e.g., IL-2. As another example, where the mammalian cell is a CD3⁺ T cell, the method can include contacting the CD3⁺ T cell with an antibody specific for CD3, where such contacting activates the CD3⁺ T cell. The antibody can be coupled to a magnetic bead.

In some cases, a method for modifying the genome of a mammalian cell comprises administering the modified cell to an individual in need thereof.

Agents that Increase the Level of a p53 Polypeptide in a Mammalian Cell

Agents that increase the level of a p53 polypeptide in a mammalian cell include, e.g., a nucleic acid comprising a nucleotide sequence encoding a p53 polypeptide; a small molecule inhibitor of MDM2; and an agent that inhibits binding of a p53 polypeptide to MDM2.

MDM2 (also known as “mouse double minute 2 homolog” and “E3 ubiquitin-protein ligase Mdm2”) is a polypeptide that promotes degradation of a p53 polypeptide in a mammalian cell.

### Nucleic Acids

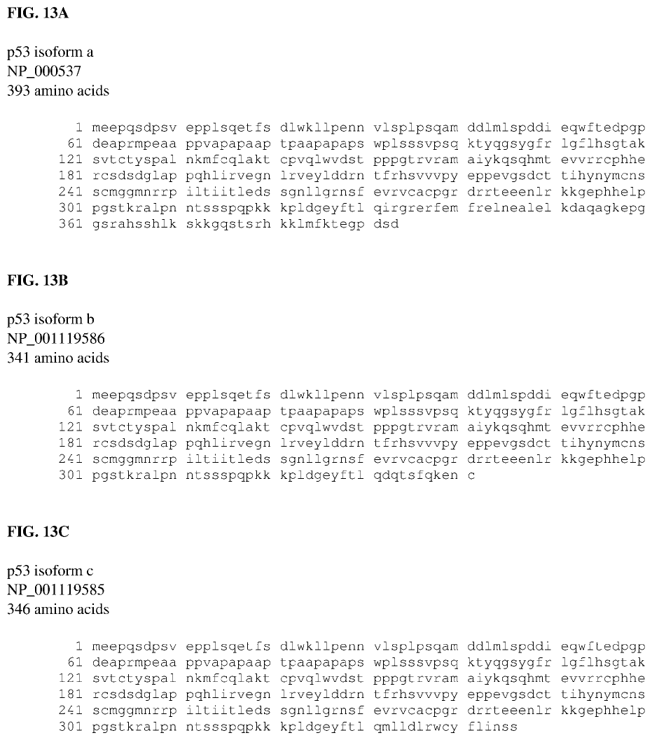

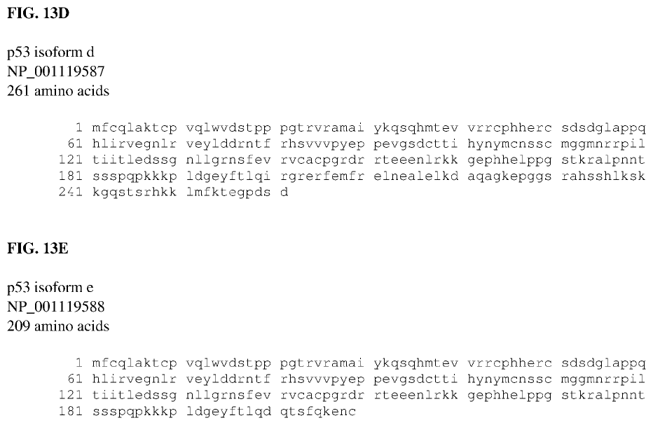

In some cases, an agent that increases the level of a p53 polypeptide in a mammalian cell is a nucleic acid comprising a nucleotide sequence encoding a p53 polypeptide. Amino acid sequences of p53 polypeptides are known in the art, and a nucleic acid comprising a nucleotide sequence encoding any such p53 polypeptide can be used. For example, in some cases, the nucleic acid comprises a nucleotide sequence encoding a p53 polypeptide having at least 85%, at least 90%, at least 95%, at least 98%, at least 99%, or 100%, amino acid sequence identity to the amino acid sequence depicted in FIG. 13A (p53, isoform a). In some cases, the nucleic acid comprises a nucleotide sequence encoding a p53 polypeptide having at least 85%, at least 90%, at least 95%, at least 98%, at least 99%, or 100%, amino acid sequence identity to any one of the amino acid sequences depicted in FIG. 13B-13E.

In some cases, the nucleic acid is an mRNA. In some cases, the nucleic acid is a recombinant expression vector comprising a nucleotide sequence encoding a p53 polypeptide. Suitable recombinant expression vectors include recombinant retroviral vectors, recombinant lentiviral vectors, recombinant adeno-associated viral vectors, recombinant adenoviral vectors, and the like.

In some cases, the nucleotide sequence encoding the p53 polypeptide is operably linked to one or more transcriptional control elements. Suitable transcriptional control elements include, e.g., constitutive promoters, regulatable promoters, and cell type-specific promoters. For example, in some cases, the promoter is a CD4⁺ T cell-specific promoter. Suitable control elements for use in CD4+ T cells include a cytomegalovirus (CMV) promoter, an elongation factor 1 (EF1) promoter, a phosphoglycerate kinase (PGK) promoter, and RPBSA (see e.g. Rad et al (2020) PLoS One 15(7): e0232915, doi: 10.1371/journal.pone.0232915). In some cases, the promoter is one that is functional in a stem cell. For example, in some cases, suitable stem cell promoters may include the human β-actin promoter (ACTB), a CMV promoter, an elongation factor-1α, (EF1α) promoter, a phosphoglycerate kinase (PGK) promoter, and a ubiquitinC (UbC) promoter (see Norrman et al (2010) PLoS One 5:e12413; doi.org/10.1371/journal.pone.0012413).

### Small Molecules

In some cases, the agent that increases the level of a p53 polypeptide in a mammalian cell is a small molecule inhibitor of MDM2. In some cases, the agent that increases the level of a p53 polypeptide in a mammalian cell is an agent that inhibits binding of an MDM2 polypeptide to a p53 polypeptide.

Small molecule inhibitors of MDM2 are known in the art, and any such inhibitor can be used. See, e.g., Zhu et al. (2022) J. Hematol Oncol. 15:91; Nguyen et al. (2017) Pharmacol. Ther. 178:92; Yu et al. (2014) Apoptosis 19:1055; and WO 2020/092221. Suitable inhibitors of MDM2 include, but are not limited to, nutlin-1, nutlin-2, nutlin-3, RG7388, RG7775, RG7112, AMG232, CGM097, HDM201, APG115, DS-3032b, MI77301, MK-8242, APR-246, RO6839921, NVP-CGM097, JNJ-26854165, RO5353, RO2468, RO8994, HLI98, SP-141, SJ-172550, XI-006, XI-011, CTX1, RO-2443, RO-05963, MI-219, MI-888, MI-773, AM-8735, AM-8553, AM-7209, AMG-232, HDM201, MK-8242, RO6839921, DS-3032, Tenovin-1/6, Leptomycin B, Inauhzin, RITA, PK083, PK7088, ZMC1, RETRA, NSC59984, Prodigiosin, P53R3, SCH529074, CP-31398, Chetomin, and TDP665759.

RG7112 has the following structure:

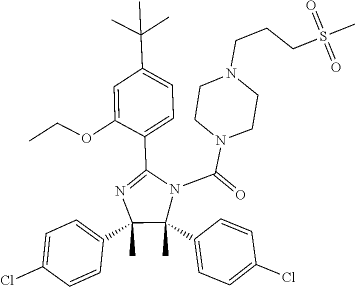 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

RG7388 has the following structure:

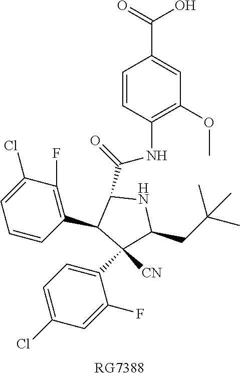 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

MK-8242 has the following structure:

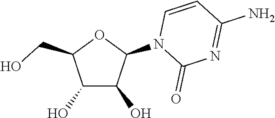 [SMILES: Nc1ccn([C@@H]2O[C@H](CO)[C@@H](O)[C@@H]2O)c(=O)n1]

AMG232 has the following structure:

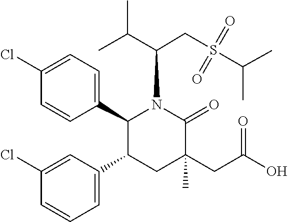 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

SAR4-5388/MI77301 has the following structure:

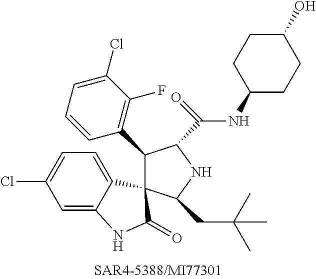 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

DS-3032b has the following structure:

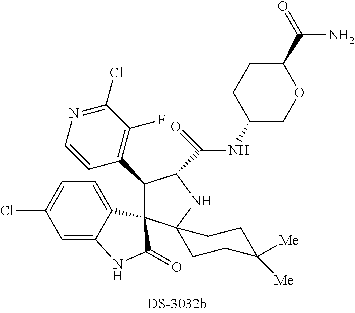 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

HDM201 has the following structure:

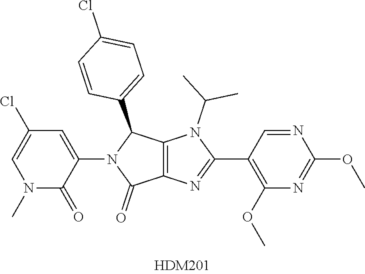 [SMILES: COc1ncc(-c2nc3c(n2C(C)C)[C@H](c2ccc(Cl)cc2)N(c2cc(Cl)cn(C)c2=O)C3=O)c(OC)n1]

NVP-CGM097 has the following structure:

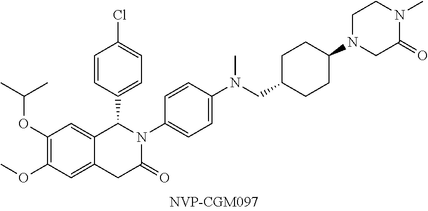 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

APG115 has the following structure:

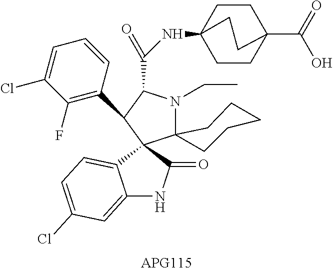

The structure of nutlin-3 is as follows:

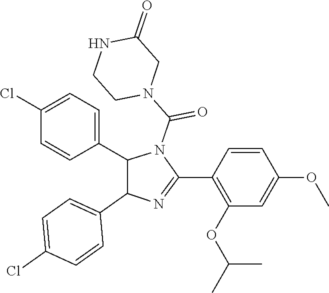 [SMILES: COc1ccc(C2=NC(c3ccc(Cl)cc3)C(c3ccc(Cl)cc3)N2C(=O)N2CCNC(=O)C2)c(OC(C)C)c1]

In some cases, a mammalian cell is present in a liquid medium in vitro, and an inhibitor of an MDM2 polypeptide is present in the liquid medium in a concentration of from about 100 nM to about 100 μM (e.g., from about 100 nM to about 500 nM, from about 500 nM to about 1 μM, from about 1 μM to about 10 μM, from about 10 μM to about 25 μM, from about 25 μM to about 50 μM, from about 50 μM to about 75 μM, or from about 75 μM to about 100 μM. In some cases, an inhibitor of an MDM2 polypeptide is present in the liquid medium in a concentration of from 10 μM to about 30 μM. In some cases, the inhibitor of an MDM2 polypeptide is nutlin-3 and is present in the liquid medium in a concentration of from 10 μM to about 30 μM.

### Gene-Editing Compositions

As noted above, a method of the present disclosure comprises contacting a mammalian cell in vitro with a gene-editing composition. A gene-editing composition comprises: a) a CRISPR-Cas effector polypeptide or a nucleic acid comprising a nucleotide sequence encoding a CRISPR-Cas effector polypeptide; and b) a guide nucleic acid or a nucleic acid comprising a nucleotide sequence encoding a guide nucleic acid. The gene editing composition can further include a donor nucleic acid, or a nucleic acid comprising a nucleotide sequence encoding a donor nucleic acid. In some cases, a gene editing composition comprises a ribonucleoprotein (RNP) comprising a CRISPR-Cas effector polypeptide and a guide RNA. In some cases, a gene editing composition comprises a base editor (e.g. Komor et al (2016) Nature. 533(7603):420-424. doi: 10.1038/nature17946). In some cases, a gene editing composition comprises a prime editor (e.g. Anzalone et al (2019) Nature 576: 149-157 https://doi.org/10.1038/s41586-019-1711-4). In some cases, a gene editing composition comprises a zinc finger nuclease (ZFN) or a transcription activator like effector nuclease (TALEN). In some cases, a gene editing composition comprises a meganuclease.

Suitable CRISPR-Cas effector polypeptides include Type II CRISPR-Cas effector polypeptides, Type III CRISPR Cas effector polypeptides, and Type VI CRISPR-Cas effector polypeptides. Examples of Type II CRISPR-Cas polypeptides include Cas9 polypeptides, e.g., Staphylococcus aureus Cas9, Streptococcus pyogenes Cas9, etc. Examples of Type VI CRISPR-Cas effector polypeptides include a Cas13a polypeptide, a Cas13b polypeptide, a Cas13c polypeptide, a Cas13d polypeptide, a Cas13e polypeptide, a Cas13f polypeptide, a Cas13X polypeptide, and a Cas13Y polypeptide. A suitable Type III CRISPR-Cas effector polypeptide is a Cas7-11 polypeptide. Suitable CRISPR-Cas effector polypeptides include fusion CRISPR-Cas effector polypeptides, e.g., comprising: i) a CRISPR-Cas effector polypeptide; and ii) a heterologous fusion partner, where suitable heterologous fusion partners include, e.g., a reverse transcriptase, a cytidine deaminase, an adenosine deaminase, an adenosine deaminase acting on RNA (ADAR) family protein, and the like.

A guide nucleic acid can include ribonucleotides and/or deoxyribonucleotides. The discussion that follows refers to a “guide RNA”; however, such discussion should be understood to include a guide nucleic acid generally. A guide RNA can be said to include two segments, a targeting segment and a protein-binding segment. The protein-binding segment is also referred to herein as the “constant region” of the guide RNA. The targeting segment of a guide RNA includes a nucleotide sequence (a guide sequence) that is complementary to (and therefore hybridizes with) a specific sequence (a target site) within a target nucleic acid (e.g., a target locus within the genome of a mammalian cell). The protein-binding segment (or “protein-binding sequence”) interacts with (binds to) a CRISPR-Cas effector polypeptide of the present disclosure. The protein-binding segment of a guide RNA can include two complementary stretches of nucleotides that hybridize to one another to form a double stranded RNA duplex (dsRNA duplex). Site-specific binding and/or cleavage of a target nucleic acid can occur at locations (e.g., target sequence of a target locus) determined by base-pairing complementarity between the guide RNA (the guide sequence of the guide RNA) and the target nucleic acid.

In some cases, the guide nucleic acid is an RNA. In some cases, the guide nucleic is a single-molecule RNA; such a guide nucleic acid is generally referred to as a “single-guide RNA” or “sgRNA.” In some cases, the guide nucleic acid is a dual-guide RNA.

In some cases, the guide nucleic acid comprises one or more modifications, e.g., a base modification, a backbone modification, etc., to provide the nucleic acid with a new or enhanced feature (e.g., improved stability).

A donor nucleic acid can encode a polypeptide of interest. A donor nucleic acid can be provided as a recombinant expression vector comprising the donor nucleic acid (or a nucleotide sequence encoding the donor nucleic acid). Polypeptides of interest include, but are not limited to, a chimeric antigen receptor (CAR), a T-cell antigen receptor (TCR), a cytokine, insulin, hemoglobin, β-globin, a neurotransmitter, a receptor, a ligand for a receptor, dystrophin, a retinal protein, cystic fibrosis transmembrane conductance regulator (CFTR), and the like. Polypeptides of interest include normal (non-mutated; functional) counterparts of diseased (e.g., mutant) proteins that have reduced function or are non-functional or that have a pathological gain of function.

### Mammalian Cells

Mammalian cells that can be modified using a method of the present disclosure include, e.g., human cells, rodent cells (mouse cells; rat cells), non-human primate cells, equine (e.g., horse) cells, bovine cells, feline cells, canine cells, and the like. In some cases, the mammalian cells are human cells.

Mammalian cells that can be modified using a method of the present disclosure include T cells (e.g., CD8⁺ T cells; CD4⁺ T cells; CD3⁺ T cells; regulatory T cells (Tregs)), B cells, natural killer (NK) cells, stem cells, myeloid cells, and the like. Suitable stem cells include, e.g., pluripotent stem cells, hematopoietic stem cells, hematopoietic stem/progenitor cells (HSPCs), induced pluripotent stem cells, and the like. Suitable mammalian cells include cells derived from iPSCs (see, e.g., Kim et al. (2022) Stem Cell Rev. Rep. 18:142). For example, suitable mammalian cells include iPSC-derived pancreatic islet cells; see, e.g., Maxwell and Millman (2021) Cell Rep. Med. 2:100238. As another example, suitable mammalian cells include iPSC-derived neuronal cells.

In some cases, the cells are patient-derived cells. In some cases, the cells are patient-derived CD3⁺ T cells. For example, in some cases, CD3⁺ T cells are obtained from a patient, modified according to a method of the present disclosure, and the modified cells are introduced into the same patient from whom the CD3⁺ T cells were obtained. For example, the CD3⁺ T cells are modified to express a CAR, using a method of the present disclosure, and the modified cells are administered to the patient from whom the CD3⁺ T cells were obtained, e.g., where the patient has a cancer that can be targeted with the CAR.

### Treatment Methods

The present disclosure provides a treatment method comprising: a) generating a modified cell by a method according to the present disclosure; and b) administering the modified cell to an individual in need thereof. Thus, the present disclosure provides a treatment method comprising: A) modifying the genome of a mammalian cell, the method comprising contacting the mammalian cell in vitro with: a) a gene editing composition comprising: i) a CRISPR-Cas effector polypeptide, or a nucleic acid comprising a nucleotide sequence encoding the CRISPR-Cas effector polypeptide; and ii) a guide nucleic acid, or a nucleic acid comprising a nucleotide sequence encoding the guide nucleic acid; and b) an agent that increases the level of a p53 polypeptide in the cell, wherein said contacting results in modification of the genome of the cell, thereby producing a modified cell; and B) administering the modified cell to an individual in need thereof. In some cases, the treatment method comprises: A) modifying the genome of a mammalian cell obtained from an individual, the method comprising contacting the mammalian cell in vitro with: a) a gene editing composition comprising: i) a CRISPR-Cas effector polypeptide, or a nucleic acid comprising a nucleotide sequence encoding the CRISPR-Cas effector polypeptide; and ii) a guide nucleic acid, or a nucleic acid comprising a nucleotide sequence encoding the guide nucleic acid; and b) an agent that increases the level of a p53 polypeptide in the cell, wherein said contacting results in modification of the genome of the cell, thereby producing a modified cell; and B) administering the modified cell to the individual from whom the mammalian cell was obtained.

In some cases, the treatment method is a method of treating cancer in an individual. In some cases, the treatment method is a method of treating cancer in an individual, wherein the modified cell is a modified T cell, NK cell, or myeloid cell that has been modified to express a CAR comprising an antigen-binding polypeptide specific for a cancer-associated antigen or a heterologous TCR specific for a cancer-associated antigen, and where the administering step treats the cancer in the individual.

In some cases, the treatment method is a method of treating diabetes (e.g., type 1 diabetes mellitus) in an individual, where the modified cell is a modified pancreatic islet cell, and where the administering step treats the diabetes in the individual.

In some cases, the treatment method is a method of treating sickle cell disease (SCD) in an individual, where the modified cell is a modified hematopoietic stem/progenitor cell (HSPC), and where the administering step treats the sickle cell disease in the individual. In some cases, the HSPC is modified to repair a mutant β-globin gene so that it encodes a normal (non-mutant, functional) β-globin.

In some cases, the treatment method is a method of treating an autoimmune disease, wherein the modified cell is a modified T cell, B cell, or Treg cell, and wherein the administering step treats the autoimmune disease. Non-limiting examples of such autoimmune diseases include systemic lupus erythematosus, Sjögren syndrome, rheumatoid arthritis, myasthenia gravis, multiple sclerosis, Hashimoto thyroiditis, and celiac disease. In some cases, the modified Treg cells are used to prevent, or reduce the likelihood of, rejection of transplanted cells, a transplanted tissue, or a transplanted organ.

In some cases, the treatment method is a method of treating a disease of the central nervous system, wherein the modified cell is a modified neuronal stem cell, and wherein the administering step treats the disease of the central nervous system. Non-limiting examples of such central nervous system diseases include Parkinson's disease, Alzheimer's disease, amyotrophic lateral sclerosis, Huntington's disease, and spinal cord injuries.

In some cases, the treatment method is a method of treating human immunodeficiency virus (HIV)/acquired immunodeficiency syndrome (AIDS), sickle cell disease, beta-thalassemia or vascular/cardiac disease, wherein the modified cell is a modified T cell, B cell, or modified hematopoietic stem cell, and wherein the administering step treats the disease.

### Gene-Editing Compositions

The present disclosure provides a gene editing composition comprising: a) a CRISPR-Cas effector polypeptide or a nucleic acid comprising a nucleotide sequence encoding a CRISPR-Cas effector polypeptide; b) a guide nucleic acid or a nucleic acid comprising a nucleotide sequence encoding a guide nucleic acid; and c) an agent that increases the level of a p53 polypeptide in a mammalian cell. A gene-editing composition of the present disclosure can further include a donor nucleic acid, or a nucleic acid comprising a nucleotide sequence encoding a donor nucleic acid. In some cases, a gene editing composition comprises a ribonucleoprotein (RNP) comprising a CRISPR-Cas effector polypeptide and a guide RNA.

Suitable CRISPR-Cas effector polypeptides, guide nucleic acids, and agents that increase the level of a p53 polypeptide are as described above. In some cases, the composition comprises a cell culture medium.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method for modifying the genome of a mammalian cell, the method comprising contacting the mammalian cell in vitro with: a) a gene editing composition comprising: i) a CRISPR-Cas effector polypeptide; and ii) a guide nucleic acid; and b) an agent that increases the level of a p53 polypeptide in the cell, wherein said contacting results in modification of the genome of the cell, thereby producing a modified cell.

Aspect 2. The method of aspect 1, wherein the agent comprises a nucleic acid comprising a nucleotide sequence encoding a p53 polypeptide.

Aspect 3. The method of aspect 2, wherein the nucleic acid is an mRNA.

Aspect 4. The method of aspect 2, wherein the nucleic acid is a recombinant expression vector.

Aspect 5. The method of aspect 4, wherein the recombinant expression vector is a lentiviral vector or an adeno-associated viral vector.

Aspect 6. The method of any one of aspects 2-5, wherein the nucleotide sequence is operably linked to a promoter that is functional in a mammalian cell.

Aspect 7. The method of aspect 1, wherein the agent is a small molecule that inhibits degradation of the p53 polypeptide in the cell, thereby increasing the level of the p53 polypeptide in the cell.

Aspect 8. The method of aspect 7, wherein the agent is an inhibitor of MDM2.

Aspect 9. The method of aspect 8, wherein the agent is selected from: nutlin-1, nutlin-2, nutlin-3, RG7388, RG7775, RG7112, AMG232, CGM097, HDM201, APG115, DS-3032b, MI77301, MK-8242, APR-246, RO6839921, NVP-CGM097, JNJ-26854165, RO5353, RO2468, RO8994, HLI98, SP-141, SJ-172550, XI-006, XI-011, CTX1, RO-2443, RO-05963, MI-219, MI-888, MI-773, AM-8735, AM-8553, AM-7209, AMG-232, HDM201, MK-8242, RO6839921, DS-3032, Tenovin-1/6, Leptomycin B, Inauhzin, RITA, PK083, PK7088, ZMC1, RETRA, NSC59984, Prodigiosin, P53R3, SCH529074, CP-31398, Chetomin, and TDP665759.

Aspect 10. The method of aspect 7, wherein the agent inhibits binding of MDM2 to p53.

Aspect 11. The method of any one of aspects 1-10, wherein the mammalian cell is a stem cell.

Aspect 12. The method of aspect 11, wherein the stem cell is a hematopoietic stem cell.

Aspect 13. The method of aspect 11, wherein the stem cell is an induced pluripotent stem cell (IPSC).

Aspect 14. The method of aspect 13, wherein the cell is a pancreatic cell derived from an IPSC.

Aspect 15. The method of any one of aspects 1-10, wherein the mammalian cell is a natural killer cell.

Aspect 16. The method of any one of aspects 1-10, wherein the mammalian cell is a B cell.

Aspect 17. The method of any one of aspects 1-10, wherein the mammalian cell is a T cell.

Aspect 18. The method of any one of aspects 1-10, wherein the mammalian cell is a myeloid cell.

Aspect 19. The method of any one of aspects 1-18, wherein the gene editing composition further comprises a donor nucleic acid.

Aspect 20. The method of aspect 19, wherein the donor nucleic acid comprises a nucleotide sequence encoding a chimeric antigen receptor.

Aspect 21. The method of aspect 19, wherein the donor nucleic acid comprises a nucleotide sequence encoding a heterologous T-cell receptor.

Aspect 22. The method of any one of aspects 1-21, wherein the step of contacting the mammalian cell with an agent that increases the level of a p53 polypeptide in the cell is carried out before the step of contacting the cell with the gene editing composition.

Aspect 23. The method of aspect 22, wherein the step of contacting the mammalian cell with an agent that increases the level of a p53 polypeptide in the cell is carried out from 1 hour to 72 hours before the step of contacting the cell with the gene editing composition.

Aspect 24. The method of any one of aspects 1-21, wherein the step of contacting the mammalian cell with an agent that increases the level of a p53 polypeptide in the cell is carried out substantially simultaneously with the step of contacting the cell with the gene editing composition.

Aspect 25. A treatment method comprising: a) generating a modified mammalian cell according to the method of any one of aspects 1-24; and b) administering the modified cell to an individual in need thereof.

Aspect 26. The method of aspect 25, wherein the treatment method is a method of treating cancer in the individual, wherein the modified cell is a modified T cell that has been modified to express a chimeric antigen receptor comprising an antigen-binding polypeptide specific for a cancer-associated antigen, and wherein said administering treats the cancer in the individual.

Aspect 27. The method of aspect 25, wherein the modified cell is a modified myeloid cell.

Aspect 28. The method of aspect 25, wherein the modified cell is a modified pancreatic islet cell, and wherein said administering treats diabetes in the individual.

Aspect 29. A gene editing composition comprising: a) a gene editing composition comprising: i) a CRISPR-Cas effector polypeptide; and ii) a guide nucleic acid; and b) an agent that increases the level of a p53 polypeptide in a mammalian cell.

Aspect 30. The gene editing composition of aspect 29, wherein the agent comprises a nucleic acid comprising a nucleotide sequence encoding a p53 polypeptide.

Aspect 31. The gene editing composition of aspect 30, wherein the nucleic acid is an mRNA.

Aspect 32. The gene editing composition of aspect 30, wherein the nucleic acid is a recombinant expression vector.

Aspect 33. The gene editing composition of aspect 32, wherein the recombinant expression vector is a lentiviral vector or an adeno-associated viral vector.

Aspect 34. The gene editing composition of any one of aspects 29-33, wherein the nucleotide sequence is operably linked to a promoter that is functional in a mammalian cell.

Aspect 35. The gene editing composition of aspect 29, wherein the agent is a small molecule that inhibits degradation of the p53 polypeptide in the cell, thereby increasing the level of the p53 polypeptide in the cell.

Aspect 36. The gene editing composition of aspect 29, wherein the agent is an inhibitor of MDM2.

Aspect 37. The gene editing composition of aspect 36, wherein the agent is selected from: nutlin-1, nutlin-2, nutlin-3, RG7388, RG7775, RG7112, AMG232, CGM097, HDM201, APG115, DS-3032b, MI77301, MK-8242, APR-246, RO6839921, NVP-CGM097, JNJ-26854165, RO5353, RO2468, RO8994, HLI98, SP-141, SJ-172550, XI-006, XI-011, CTX1, RO-2443, RO-05963, MI-219, MI-888, MI-773, AM-8735, AM-8553, AM-7209, AMG-232, HDM201, MK-8242, RO6839921, DS-3032, Tenovin-1/6, Leptomycin B, Inauhzin, RITA, PK083, PK7088, ZMC1, RETRA, NSC59984, Prodigiosin, P53R3, SCH529074, CP-31398, Chetomin, and TDP665759.

Aspect 38. The gene editing composition of aspect 29, wherein the agent inhibits binding of MDM2 to p53.

Aspect 39. The gene editing composition of any one of aspects 29-38, further comprising a donor nucleic acid.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

CRISPR-Cas9 genome editing has enabled advanced T cell therapies, but occasional loss of the targeted chromosome remains a safety concern. To investigate whether Cas9-induced chromosome loss is a universal phenomenon and evaluate its clinical significance, a systematic analysis in primary human T cells was conducted. Arrayed and pooled CRISPR screens revealed that chromosome loss was generalizable across the genome and resulted in partial and entire loss of the chromosome, including in pre-clinical chimeric antigen receptor T cells. T cells with chromosome loss persisted for weeks in culture, implying the potential to interfere with clinical use. A modified cell manufacturing process dramatically reduced chromosome loss while largely preserving genome editing efficacy. Expression of p53 correlated with protection from chromosome loss observed in this protocol, suggesting both a mechanism and strategy for T cell engineering that mitigates this genotoxicity in the clinic.

### Methods

### Cell Culture

Primary adult peripheral blood mononuclear cells (PBMCs) were obtained as cryopreserved vials from Allcells Inc. CD3+ T cells were isolated from PBMCs using EasySep Human T Cell Isolation Kits (StemCell Technologies) according to the manufacturer's instructions. Isolated CD3+ T cells were cultured in X-Vivo 15 medium (Lonza) with 5% fetal bovine serum (FBS) (VWR), 50 μM 2-mercaptoethanol, and 10 mM N-acetyl L-cysteine (Sigma-Aldrich). One day post-isolation, CD3+ T cells were activated and stimulated with a 1:1 ratio of anti-human CD3/CD28 magnetic Dynabeads (Thermo Fisher) to cells, as well as 5 ng/mL IL-7 (PeproTech), 5 ng/mL IL-15 (R&D Systems), and 300 U/mL IL-2 (PeproTech) for three days. After the initial activation and stimulation, magnetic beads were removed and T cells were cultured in medium with 300 U/mL IL-2. Medium was replaced every other day and T cells were maintained at a density of ~0.5-1×10⁶ cells/mL.

For CAR T cell experiments, primary adult PBMCs were obtained as Leukopaks (StemCell Technologies) from deidentified healthy donors and cryopreserved in RPMI medium supplemented with 20% human serum and 10% DMSO. T cells were isolated as described before and cultured in X-Vivo 15 medium supplemented with 5% human serum, 5 ng/mL IL-7 (Miltenyi Biotec), and 5 ng/mL IL-15 (Miltenyi Biotec). Immediately after isolation, T cells were stimulated for two days with anti-human CD3/CD28 magnetic Dynabeads (Thermo Fisher) using a 1:1 bead-to-cell ratio.

### Cas9 Ribonucleoprotein Nucleofection

100 pmol Alt-R crRNA and 100 pmol Alt-R tracrRNA (IDT) were diluted in IDT Duplex Buffer, incubated at 90° C. for 5 min, and then slow cooled to room temperature (Supplementary Tables 1, 7-8). 50 pmol S. pyogenes Cas9 V3 (IDT) was diluted in RNP buffer (20 mM HEPES, 150 mM NaCl, 10% Glycerol, 1 mM MgCl₂, pH 7.5). Cas9 and duplexed gRNA (1:2 molar ratio) were incubated at 37° C. for 15 min. Primary human T cells were washed once with PBS(−/−) before 250,000 cells were resuspended in P3 Buffer (Lonza). 50 pmol Cas9 RNP was added to the cells before nucleofection in a Lonza 4D-Nucleofector with pulse code EH-115. X-Vivo 15 medium with 300 U/mL IL-2 was added to the nucleofected cells before a 30 min recovery at 37° C. Nucleofected T cells were plated at a density of ~0.5-1×10⁶ cells/mL in 96-well U-bottom plates.

### TCR Flow Cytometry

T cells were resuspended in Cell Staining Buffer (BioLegend) with Ghost Dye Red 780 (1:1,000, TonboBio) and anti-human TCR α/β Brilliant Violet 421 (1:100, BioLegend). Cells were stained for 30 min at 4° C. in the dark. After staining, cells were washed with Cell Staining Buffer and analyzed on a Thermo Fisher Attune NXT Flow Cytometer with an autosampler. Over 20,000-100,000 cells were routinely collected and analyzed with FlowJo.

### Next-Generation Sequencing of TRAC Genome Editing

Genomic DNA from T cells was extracted with QuickExtract DNA Extraction Solution (Lucigen) by incubating resuspended cells for 10 minutes at room temperature before heating lysates at 65° C. for 20 minutes then 95° C. for 20 min. The region of TRAC containing the Cas9 target site was amplified from genomic DNA with Q5 High-Fidelity DNA polymerase (NEB) to add universal adaptors (Supplementary Table 2). Amplicons were cleaned with SPRIselect beads (Beckman Coulter) before a second round of PCR was performed to add unique i5 and i7 Illumina indices to each sample. Subsequent amplicons were cleaned again, and libraries were sequenced on an Illumina iSeq 100 (2×150 bp). FASTQ files were trimmed, merged, and analyzed for indels with CRISPResso2 (crispresso2.pincllolab.org).⁵⁸ For non-targeting conditions, sequencing from cells receiving Cas9 and a non-targeting gRNA was used as input to check for indels at a given region.

Single-Cell RNA Sequencing with MULTI-Seq Barcoding

Four days post-nucleofection, T cells were labeled via MULTI-seq as previously described.¹⁵ Briefly, a lipid-modified oligonucleotide (LMO) was combined with a unique oligonucleotide barcode (Supplementary Tables 3 and 6) at a 1:1 molar ratio in PBS(−/−). 500,000 cells or fewer were washed twice with PBS(−/−) and then resuspended in PBS(−/−). The LMO/barcode solution was mixed with each cell suspension and incubated on ice for 5 minutes before addition of a co-anchor LMO and incubation on ice for an additional 5 minutes. Cold 1% BSA in PBS(−/−) was added to sequester free LMOs before washing cells twice with cold 1% BSA in PBS(−/−). Uniquely labeled cells were pooled in equal numbers in 1% BSA in PBS(−/−) to a final concentration of ~1,600 cells/mL. 10× Genomics Chromium Next GEM Single Cell 3′ Gene Expression Kits (v3.1) were utilized according to the manufacturer's instructions, with the following modifications. Lanes of a standard Chromium chip were “super loaded” with ~50,000 cells to yield a target cell recovery of ~25,000 cells. During the cDNA amplification, 1 μL of a 2.5 μM MULTI-seq primer (see McGinnis et al.) was added. Supernatant from the cDNA bead cleanup was saved because it contained the MULTI-seq barcode amplicon. Supernatants were further cleaned by addition of SPRIselect beads and isopropanol with a conventional magnetic bead cleanup protocol. 3.5 ng of each cleaned amplicon was used in a PCR reaction to add sequencing indices; the reactions included KAPA HiFi HotStart ReadyMix (Roche), a unique i5 primer, and a unique RPI i7 primer (Supplementary Table 3). The PCR reactions were cleaned with SPRIselect beads before final library QC. Gene expression and MULTI-seq barcode libraries were pooled 6:1 (molar ratio) and sequenced on an Illumina NovaSeq 600 S1 Flow Cell.

### Single-Cell RNA Sequencing Analysis

Cell Ranger (v7.0) was used to process Chromium single cell data. cellranger count was performed with the parameters --rl-length=28 and --r2-length=90. For the first TRAC-targeting experiment the --force-cells parameter was set to 15,000 cells. To demultiplex the different pools of cells using the MULTI-seq barcode, cellranger multi was performed. The results from the different pools were aggregated using cellranger aggr. The results from cellranger were parsed with scanpy and converted to a h5ad file format. The demultiplexed results were added as metadata to the h5ad file.

For the CROP-seq screen, the number of reads within each cell aligning to each of the gRNAs used in the screen was counted. To determine which cells were targeted by a single, unique gRNA, it was tested whether the gRNA with the highest number of reads had significantly more reads than the second highest. Specifically, let c₁ and c₂ be the number of reads for the first and second most common gRNAs in a cell, respectively. To test whether c₁ is significantly greater than c₂, a P-value was calculated based on a binomial distribution with parameters n=c₁+c₂ and p=0.5 (i.e. x~B(c₁+c₂, 0.5)). If the probability of x≥c₁ was smaller than 0.05, the cell was determined to be transduced by a single gRNA.

Quantification of Chromosome Loss from scRNA-Seq

To assess the dosage of each gene in each cell, inferCNV of the Trinity CTAT project (https://github.com/broadinstitute/infercnv) was executed in R (version 4.1) with default parameters over the h5ad dataset created by cellranger (see previous section) for every scRNA-seq dataset.¹⁶ Each cell with a gRNA was labeled as a “treatment” and each cell with a non-targeted gRNA was labeled as a “control.” To successfully run inferCNV for the CROP-seq screen, inferCNV was performed in multiple batches of 30,000 cells.

The output of inferCNV was the estimated dosage for each gene; according to the software's specifications, values below 0.95 were considered loss of at least one copy of the gene. inferCNV values for each gene were binarized as <0.95 or ≥0.95. Each chromosome in each cell was then searched for the interval between two genes that maximizes the difference between the average binarized inferCNV values on either side of the interval. This interval was the candidate breakage point for a particular chromosome in a cell.

The inferCNV values were used for all genes on a given chromosome within each cell (with respect to each of the 22 somatic chromosomes) to estimate the loss status of that chromosome. Specifically, whether there was 1) no chromosome loss, 2) whole chromosome loss, or 3) partial chromosome loss was estimated. If at least 70% of a minimum 150 genes to either the left or right of the candidate breakage point were below the 0.95 threshold, but less than 70% of the genes on the other side were below the threshold, the cell was labeled as partial chromosome loss for that chromosome. Otherwise, if at least 70% of all the genes throughout the entire chromosome were below the threshold, the cell was labeled as having whole chromosome loss for that chromosome. If neither were true, the cell was labeled as having no chromosome loss for that chromosome.

Downsampling of the CROP-seq screen dataset was performed to assess the dependency of chromosome loss enrichment on the number of genes in the inferCNV output. To do this, 4,000 or 1,000 genes contained within the 9,639 gene output of the full CROP-seq inferCNV output were randomly sampled. The chromosome loss calling pipeline, as described above, was then performed on these downsampled datasets.

### Droplet Digital Quantitative PCR

Genomic DNA was collected from T cells at different times post-nucleofection with QuickExtract DNA Extraction Solution, identical to as described earlier. The ddPCR setup was similar to what has been previously described.³⁷ For multiplexed ddPCR, two ~200 bp amplicons for each target gene were designed (Supplementary Tables 4, 7, and 8). Amplicon 1 was located proximal to the centromere and utilized a hexachlorofluorescein-labeled (HEX) oligonucleotide probe (PrimeTime qPCR probes, Zen double quencher, IDT). Amplicon 2 was located ~100-200 bp away from amplicon 1, was distal relative to the centromere, and utilized a 6-fluorescein-labeled (FAM) oligonucleotide probe (PrimeTime qPCR probes, Zen double quencher, IDT). Amplicon 1 served as a control, which should be unaffected by Cas9 genome editing or chromosome loss and would signal whether the gene of interest was in a given droplet. Amplicon 2 spanned the Cas9 target site, with the probe located ~30-60 bp away from the cleavage site. If the target site was not successfully repaired after Cas9 cleavage, amplicon 2 would not be able to be amplified and the FAM probe would be unable to dissociate from its quencher. ddPCR reactions were assembled with ddPCR Supermix for Probes (No dUTP, Bio-Rad), 900 nM of each primer, 250 nM of each probe, and 10-30 ng of genomic DNA. Droplets were formed using a Bio-Rad QX200 Droplet Generator following the manufacturer's instructions before thermal cycling. The following day, ddPCR droplets were analyzed on a Bio-Rad QX200 Droplet Reader. Data were analyzed with the QX Manager Software (Bio-Rad), and thresholds were set manually based on wells with untreated samples. The percentage of alleles with chromosome loss was calculated based on droplets that had the target amplicon 1 (HEX+) but were unable to produce the neighboring amplicon 2 (FAM−). The equation utilized is as follows:

$$\% Chromosome loss = 100 \times ( 1 - \frac{[ FAM ]}{[ HEX ]} ) .$$

### Genome-Scale CROP-Seq CRISPR Screen Design

The CROP-seq library was designed to contain multiple gRNAs that target multiple genes on every chromosome. When possible, validated gRNA sequences from previous publications were utilized (Supplementary Table 5). The gRNA library was ordered as an oPool oligo pool (IDT) and Golden Gate cloned into a custom CROP-seq vector that co-expressed GFP. To analyze the library, primers were used to amplify the gRNA spacer from either the plasmid library or genomic DNA library before sequencing on an Illumina iSeq. MAGeCK (https://sourceforge.net/p/mageck/wiki/Home/) was used to quantify the representation of each gRNA in the library.⁵⁹

### CROP-Seq CRISPR Screen Lentiviral Production

For lentivirus production, Human Embryonic Kidney 293T (HEK293T) cells were cultured in Dulbecco's Modified Eagle Medium (Gibco) with 10% FBS and 1% penicillin/streptomycin (Gibco). HEK293 Ts were transfected at 70-90% confluency with 10 μg CROP-seq gRNA plasmid, 10 μg Gag-pol expression plasmid (psPax2, gift from Didier Trono, Addgene plasmid #12260), and 1 μg pCMV-VSV-G plasmid (gift from Bob Weinberg, Addgene plasmid #121669) using polyethylenimine (PEI, Polysciences Inc.) at a 3:1 PEI:plasmid ratio. Approximately 6-8 hours after transfection, the medium was aspirated from cells and replaced with Opti-Mem (Gibco). Supernatant containing lentivirus was collected 48 hours after transfection, the medium was replaced, and medium was collected once more after an additional 48 hours. Viral supernatants were filtered through a 0.45 μm PES membrane bottle top filter (Thermo Fisher) and then concentrated with Lenti-X Concentrator (Takara) according to the manufacturer's instructions. Purified and concentrated lentivirus was used immediately or stored at −80° C. Lentivirus was titered by counting the number of initially transduced cells, adding serial dilutions of lentivirus to primary human T cells, and measuring the percentage of GFP+ cells after three days (only in conditions with <30% GFP+ cells to ensure a majority were single transduction events).

### CROP-Seq CRISPR Screen

For the CROP-seq screen, primary human T cells were isolated and stimulated as stated previously. 24 hours after stimulation, lentivirus was added to the cells at a multiplicity of infection (MOI) of ~0.3. MOI was confirmed via flow cytometry two days later, at day three post-stimulation. Dynabeads were then removed from T cells and Cas9 was nucleofected as stated previously. For the full CROP-seq library experiment, four days post-nucleofection, T cells were subject to fluorescence-activated cell sorting (FACS) on a Sony SH800S cell sorter to enrich for GFP+ cells. Genomic DNA was harvested from a small number of cells, as previously described, to assess the library representation. The rest of the live/GFP+ cells were arbitrarily divided into six pools and subject to MULTI-seq barcoding and 10× Genomics scRNA-seq, as previously described. The CROP-seq gRNA was enriched from the resulting cDNA similar to what has been previously described (Supplementary Table 6).²⁰ Briefly, 25 ng of cDNA was added to eight separate KAPA HiFi HotStart ReadyMix PCR reactions and amplified for 10 cycles with an annealing temperature of 65° C. to enrich for the gRNA. Individual PCR reactions were pooled together and cleaned with SPRIselect beads. 8 μL of cleaned PCR1 product was added to a second KAPA HiFi HotStart ReadyMix PCR reaction and amplified for 10 cycles with an annealing temperature of 65° C. to add Illumina sequencing adaptors. Gene expression, MULTI-seq barcode, and CROP-seq enrichment libraries were sequenced on an Illumina NovaSeq 600. Multiple iterations of library sequencing were concatenated to achieve the desired sequencing depth.

### Strand and MMEJ Analyses

Each gRNA was mapped using the GRCh38 genome assembly. A two-sided Fisher's Exact Test was performed to determine whether gRNAs binding distal or proximal to the centromere, relative to the gRNA spacer sequence, affected chromosome loss. MMEJ analyses were performed using inDelphi (indelphi.giffordlab.mit.edu).⁶⁰ The cell type was set to K562s and the MMEJ strength was measured for each unique gRNA sequence.

### Differential Gene Expression Analysis

To identify genes differentially expressed between cells with and without chromosome loss, the memento algorithm with default parameters (capture rate=0.07) was used.⁶¹ Each gene was tested for differential expression with respect to each of the 22 somatic chromosomes separately; and only genes that were consistently over- or underexpressed across most chromosomes were reported. It was ensured that the differences that were observed were specific to the tested gene and were not the result of overall lower or greater levels of gene expression in cells with chromosome loss. To do this, the total expression of transcripts sharing the same chromosome as the tested gene was accounted for by including the overall count of all transcripts on that chromosome as a covariate. This covariate was defined with respect to the chromosome containing the gene tested for differential expression and not with respect for the chromosome determining the two compared groups (cells with or without loss of that chromosome). Since memento supports only discrete covariates, the total transcript count was discretized into 10 decile bins.

The results of memento were corrected for multiple testing using FDR over the combined set of all tested genes over all tested chromosomes. A gene was considered to be statistically significant with respect to a chromosome (i.e. the gene to be over- or underexpressed in cells losing that chromosome) if its corrected P-value was below 0.05. Accordingly, the significance status of that gene-chromosome combination was assigned to be 1, 0, or −1 if it was significantly overexpressed, not significant, or under expressed, respectively. Each gene was then a total score between −22 and 22 by summing the significance status of that gene with respect to each of the 22 somatic chromosomes. 613 genes obtained a total score ≥5 and were considered the top overexpressed genes, while 590 obtained a total score ≤−5 and were considered the top underexpressed genes.

Pathways enriched among the 613 top overexpressed genes were identified by searching through the pathway terms defined in the KEGG database using the GSEApy Python package.^{62,63}

### Cell Cycle Analysis

Cell cycle states were defined using data and methods as previously described.⁶⁴

### Epigenetic Analyses

Datasets (ENCFF233TCT, ENCFF055FYI, and ENCFF129GAM) corresponding to activated T cells from a male donor (43 years old) were selected from the ENCODE Portal (www.enccodeproject.org).^{65,66} The presence of open chromatin from ATAC-seq data and the location of epigenetic marks (H3K9me3 and H3K36me3) from ChIP-seq data were determined within a 75 bp window around the GRCh38 coordinates of each gRNA and a P-value <10⁻⁵, according to best practices.⁶⁷

### T Cell Proliferation Tracking

After isolation and stimulation, primary human T cells were nucleofected with Cas9 RNPs identical to what was described earlier. Immediately after nucleofection recovery, cells were pelleted and resuspended in 5 μM CellTrace Violet (Invitrogen). Cells were incubated in CellTrace Violet for 20 min at 37° C., prior to diluting in 4× volume of complete X-Vivo 15 medium to absorb unbound dye and incubating again for 5 min at 37° C. Cells were pelleted and resuspended in complete X-Vivo 15 with 300 U/mL IL-2. T cells were passaged every other day to refresh medium and maintain a density of ~0.5-1×10⁶ cells/mL. Cells were sorted on a BD FACSAria II to obtain the approximate bottom and top quartile of cells according to CellTrace Violet signal.

### Cas9-Mediated CD5 Homology-Directed Repair

HA tag insertion was achieved with either a single-stranded DNA HDR template (ssDNA HDRT), a double-stranded DNA HDR template (dsDNA HDRT), or a single-stranded DNA HDR template with Cas9 target sequences (ssCTS HDRT) (Supplementary Note 3).³⁹ Equimolar HDRT oligonucleotides were diluted in IDT Duplex Buffer, heated to 95° C. for 5 minutes, then allowed to slow cool to room temperature. 100 pmol of HDRT was added to Cas9 RNP nucleofections of primary human T cells, identical to as described above. Cells were analyzed on a Thermo Fisher Attune NXT Flow Cytometer with an autosampler, identical to as described above, except with the antibodies anti-human CD5 (UCHT2)-PE (1:100, Invitrogen) and anti-HA (6E2)-AF647 (1:100, Cell Signaling Technology). Over 20,000-100,000 cells were routinely collected and analyzed with FlowJo.

### Fluorescence-Activated Cell Sorting of CD5, CD81, and CD3E

Primary human T cells were nucleofected with Cas9 RNPs targeting CD5 identical to what was described before. Seven days post-nucleofection, cells were stained with anti-human TCR α/β Brilliant Violet 421 (BioLegend), anti-human CD5 (UCHT2)-PE (Invitrogen), anti-human CD81 (5A6)-FITC (BioLegend), and anti-human CD3E (SK7)-APC (Invitrogen), all at a 1:100 dilution. Cells were sorted on a BD FACSAria II to isolate different populations (Supplementary Note 1).

### CAR Adeno-Associated Virus Production

An AAV transgene plasmid encoding the inverted terminal repeats, a 1928ζ CAR, a truncated human EGFR (EGFRt) tag, and TRAC homology arms for HDR was used as previously described (Supplementary Note 4).⁴¹ The AAV plasmid was packaged into AAV6 by transfection of HEK293T cells together with pHelper and pAAV Rep-Cap plasmids using PEI. The AAVs were purified using iodixanol gradient ultracentrifugation. The titration of the AAV was determined by quantitative PCR on DNaseI (NEB) treated and proteinase K (Qiagen) digested AAV samples, using primers against the left homology arm. The quantitative PCR was performed with SsoFast EvaGreen Supermix (Bio-Rad) on a StepOnePlus Real-Time PCR System (Applied Biosystems).

### CAR T Cell Production

gRNAs targeting exon 1 of the TRAC locus (TRAC gRNA 12), the intron preceding the TRAC locus (TRAC gRNA 13), or a non-targeting control gRNA were purchased from Synthego and resuspended in TE buffer (Supplementary Table 9). Cas9 RNP was generated by incubating 60 pmol of Cas9 protein with 120 pmol sgRNA. T cells were counted, resuspended in P3 buffer at 2×10⁶ per 20 μL, mixed with 3 μL of RNPs and added to a 96-well nucleofection plate. Cells were electroporated using a Lonza 4D-Nucleofector 96-well unit with the EH-115 protocol and immediately recovered by adding pre-warmed X-Vivo 15 medium without human serum. Recombinant AAV6 encoding the HDR template was added to the culture 30 to 60 min after nucleofection at an MOI of 10⁵, and incubated with the cells overnight. The day after the nucleofection and transduction, edited cells were resuspended in medium and expanded using standard culture conditions, keeping a density of 10⁶ cells/mL. TCR disruption and CAR HDR efficiency was evaluated by flow cytometry by staining the TCR with anti-TCRα/β (BW242/412)-PE (1:50, Miltenyi) and the CAR with goat anti-mouse IgG (H+L) AlexaFluor 647 Fab (1:100, Jackson ImmunoResearch).

CAR T Cell scRNA-Seq

CAR T cells were harvested at two time points after independent nucleofections (day four and day seven post-nucleofection). TotalSeq-A0251-1 anti-human Hashtag reagents (BioLegend) were used to label different cell conditions. For the experiment, 500,000 cells from each condition were labeled with the hash antibodies in Cell Staining Buffer at 4° C. for 30 min. After labeling, cells were washed three times with Cell Staining Buffer at 4° C. and then resuspended in PBS(−/−) containing 0.04% BSA. Labeled cells were pooled and 50,000 cells were “super loaded” into four lanes (two lanes for day four samples and the other two lanes for day seven samples) of a 10× Chromium Single-Cell G Chip. A 10× Genomics Chromium Next GEM Single Cell 3′ Gene Expression Kit (v3.1) was utilized according to the manufacturer's instructions, and the subsequent library was sequenced on an Illumina NovaSeq 600 S4 Flow Cell.

### Laboratory Versus Clinical T Cell Manufacturing

For the laboratory protocol, T cells were activated and stimulated identical to what was described earlier. After nucleofection of Cas9 RNP, T cells were cultured in X-Vivo 15 medium with 5 ng/mL IL-7, 5 ng/mL IL-15, and 300 U/mL IL-2.

For the clinical protocol, a protocol similar to what was used in a phase 1 clinical trial¹ was followed. After T cell isolation, cells were cultured in X-Vivo 15 medium with 5 ng/mL IL-7 and 5 ng/mL IL-15 for two days. Non-activated T cells were nucleofected with 50 pmol Cas9 RNP using a Lonza 4D-Nucleofector with pulse code EH-115. After nucleofection, cells were incubated in X-Vivo 15 medium with 5 ng/mL IL-7 and 5 ng/mL IL-15 for a 30 min recovery at 37° C. Nucleofected T cells were plated at a density of ~0.5-1×10⁶ cells/mL in 96-well U-bottom plates. Two days after nucleofection, cells were counted and activated/stimulated with a 1:1 ratio of anti-human CD3/CD28 magnetic Dynabeads to cells, as well as 5 ng/mL IL-7, 5 ng/mL IL-15, and 300 U/mL IL-2 for an additional three days.

### T Cell RT-qPCR

During the laboratory or clinical T cell manufacturing protocols, 500,000 cells were periodically pelleted and resuspended in TRIzol (Invitrogen). RNA was isolated via phenol-chloroform extraction, precipitated by addition of isopropanol, washed with 75% ethanol, and resuspended in nuclease-free water. Isolated RNA was treated with TURBO DNase (Invitrogen) and SUPERase-In RNase inhibitor (Thermo Fisher) for 30 min at 37° C. before addition of DNase Inactivation Reagent according to the manufacturer's instructions. DNA-free RNA underwent cDNA synthesis using SuperScript III Reverse Transcriptase (Invitrogen) and Random Primers (Promega) according to the manufacturer's instructions. qPCRs were performed with the resulting cDNA using iTaq Universal SYBR Green Supermix (Bio-Rad) on a Bio-Rad CFX96 Real-Time PCR Detection System (Supplementary Table 10). TP53 expression levels were normalized to the expression levels of the housekeeping gene GAPDH, and to timepoint A (where the laboratory and clinical protocols start identically) using the ΔΔCt method.

### Results

### Single-Cell RNA Sequencing Reveals Chromosome Loss in Cas9-Edited T Cells

The TRAC locus, which encodes the T cell receptor (TCR) responsible for CD4 and CD8 T cell reactivity to peptide-MHC complexes, is of immense interest for genome editing applications in human T cells. For engineered adoptive T cell therapies, where T cells are armed with a transgenic receptor for targeted immunotherapy, disrupting endogenous TCR expression limits graft-versus-host toxicity associated with mispairing of transgenic and endogenous TCRs.¹³ Additionally, abrogating the TCR is an important step towards developing allogeneic “off-the-shelf” T cell therapies that could reduce cell manufacturing costs and expand patient accessibility.¹⁴

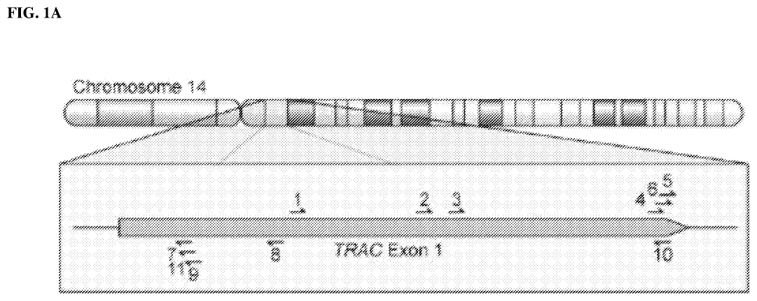

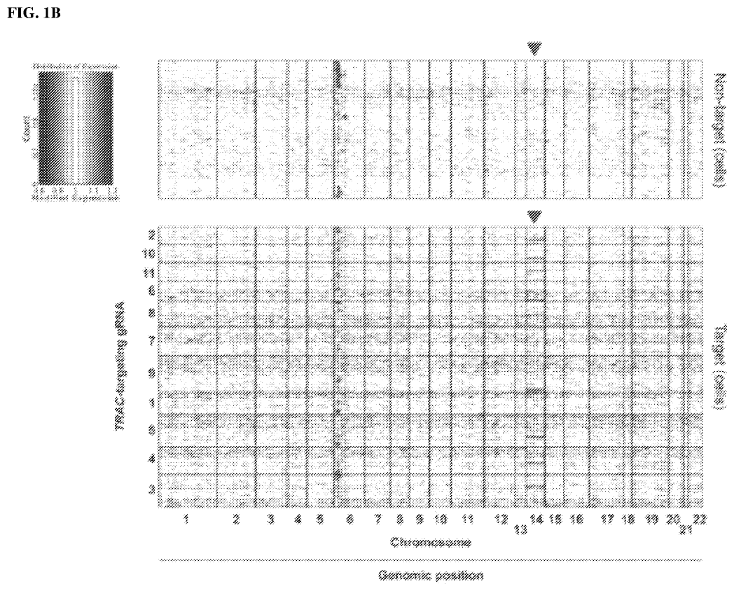

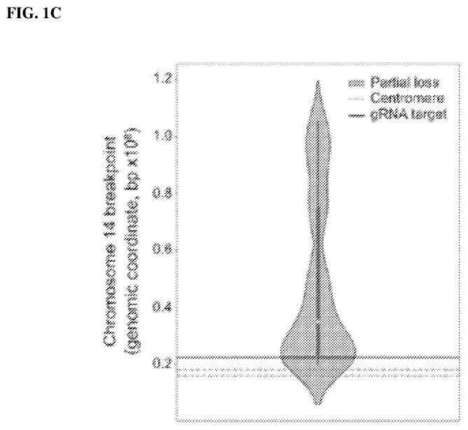

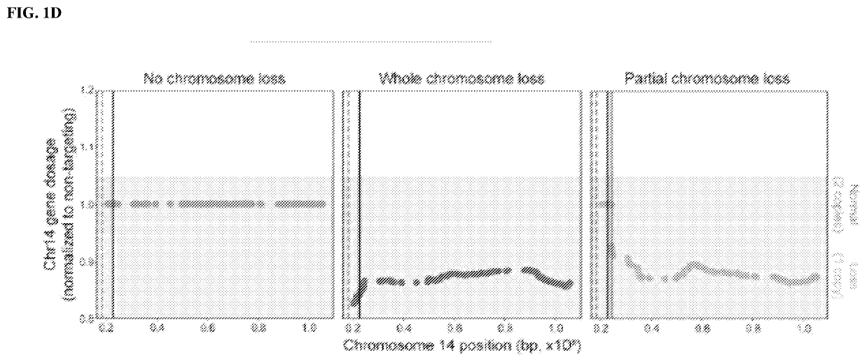

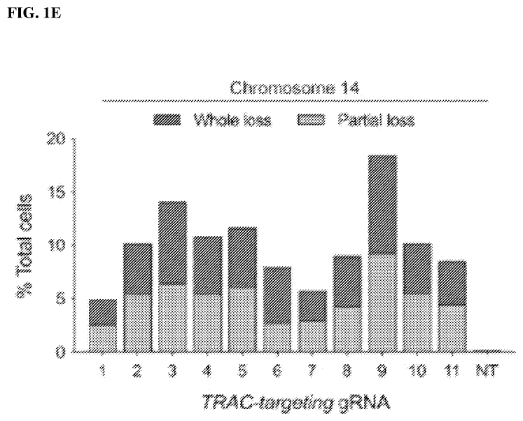

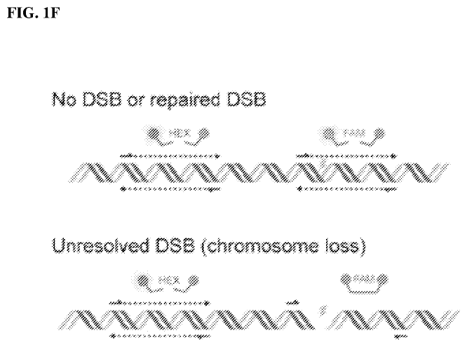

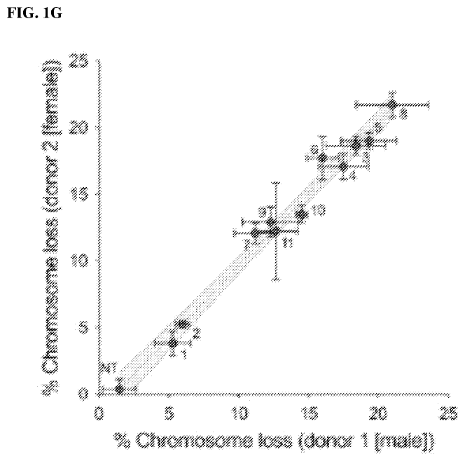

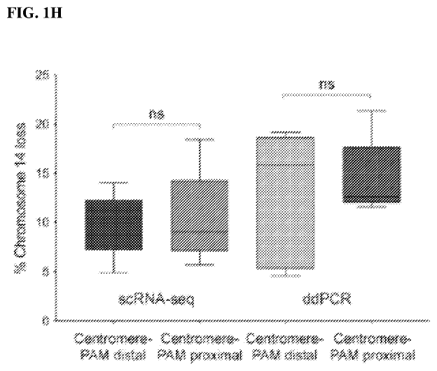

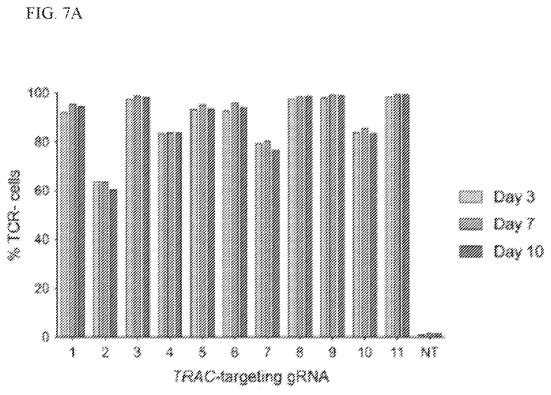

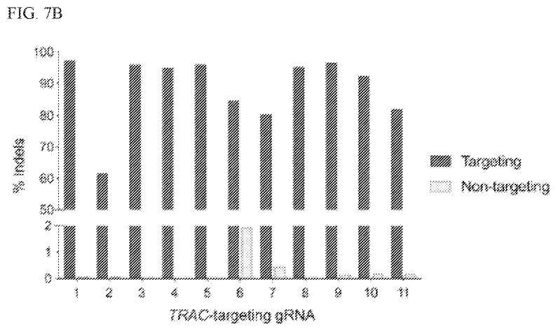

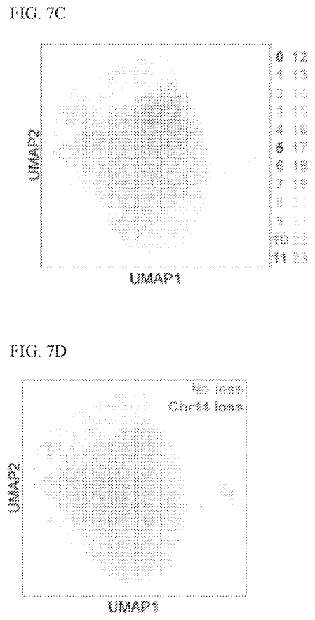

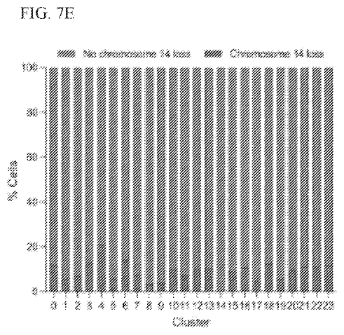

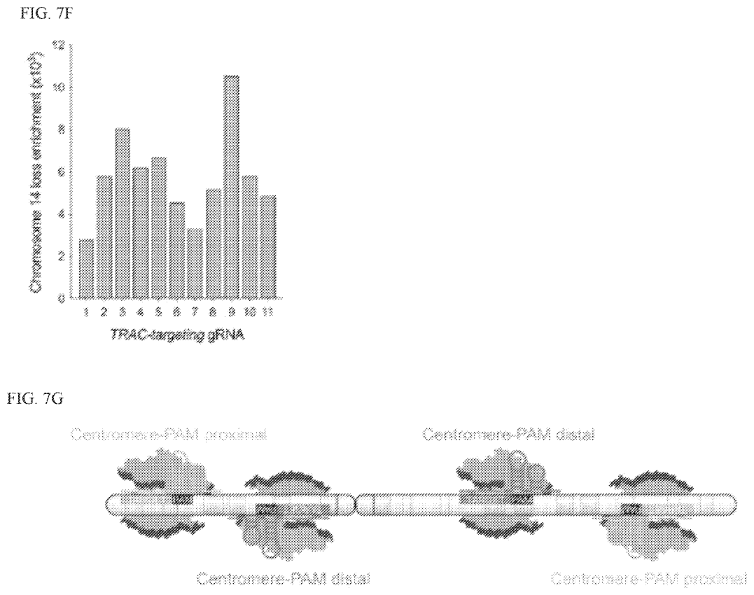

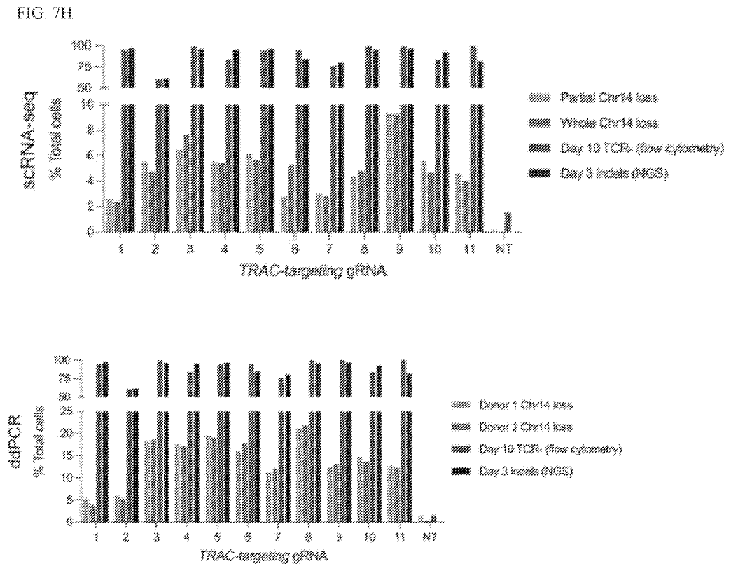

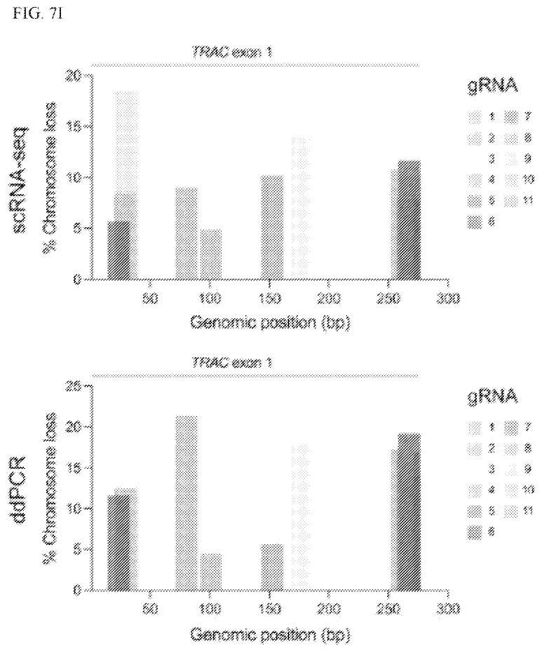

To quantify chromosome loss after genome editing of TRAC, primary human T cells were nucleofected with S. pyogenes Cas9 ribonucleoprotein (RNP) including one of 11 different gRNAs tiled across the first exon of TRAC or a non-targeting gRNA (FIG. 1A). Reduced TCR expression occurred in 60-99% of all cells as measured by flow cytometry (FIG. 7A) and editing at the TRAC locus was observed in 62-97% of all genomic DNA sequencing reads (FIG. 7B), depending on the gRNA. Four days after Cas9 RNP introduction, T cells were subjected to multiplexed single-cell RNA sequencing (scRNA-seq) to detect reduced transcript levels resulting from chromosome loss caused by Cas9-mediated double-strand DNA breaks (DSBs) (FIG. 7C).¹⁵ Transcriptome-wide analysis using existing computational methods revealed a reduction in gene dosage, specifically on chromosome 14, in cells with a TRAC-targeting gRNA compared to cells with a non-targeting gRNA (FIG. 1B).¹⁶ The distribution of breakpoint loci across chromosome 14 was estimated; the highest frequency was found to be near the known genomic location of the TRAC-targeting gRNAs (FIG. 1C). Cells with lower gene dosage originating at the Cas9 target site (partial chromosome loss), as well as cells with lower gene dosage across the entire chromosome (whole chromosome loss) (FIG. 1D), were observed. Overall, ~5-20% of T cells exhibited partial or whole loss of chromosome 14 depending on the TRAC-targeting gRNA (FIGS. 1E, 7D, 7E, and 7F).

### DNA-Based Droplet Digital PCR is an Orthogonal Method to Detect Chromosome Loss

As an orthogonal approach to detect chromosome loss, droplet digital PCR (ddPCR) was used to directly quantify genomic DNA copy number, eliminating potential interference from transcriptional or epigenetic factors that may affect the scRNA-seq results. Two primer/probe sets were designed to amplify nearby regions of the target gene, one as a control (HEX) and the other spanning the Cas9 gRNA target site (FAM) so that a DSB and chromosome loss would inhibit amplicon generation (FIG. 1F). Three days after Cas9 nucleofection, genomic DNA harvested from TRAC-targeted T cells resulted on average in ~4-22% chromosome loss; these losses were highly reproducible across biological T cell donors (FIG. 1G). Importantly, primers and probes in the amplicon spanning the intended Cas9 target site were positioned to avoid binding site disruption by small insertions and deletions (indels), the most common outcome after Cas9 genome editing.

Based on the observation that Cas9 preferentially remains bound to the non-protospacer adjacent motif (PAM) side of the target DNA after cleavage,^{17,18} it was asked whether orientation of the PAM relative to the centromere influenced chromosome loss (FIG. 7G). However, no significant difference was found between targets where the PAM was distal or proximal to the centromere, relative to the gRNA spacer sequence (FIG. 1H). Furthermore, the rates of chromosome 14 loss measured by scRNA-seq or ddPCR did not correlate with the efficacy of TCR disruption or genomic position targeted by different gRNAs (FIGS. 7H and 7I).

### Target-Specific Chromosome Loss is a General Phenomenon Following Genome Editing

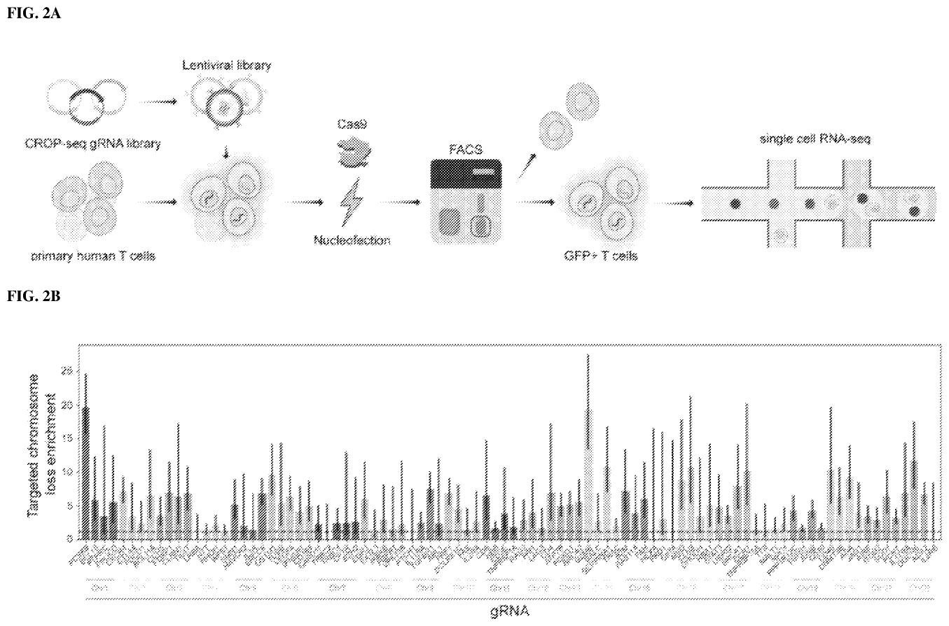

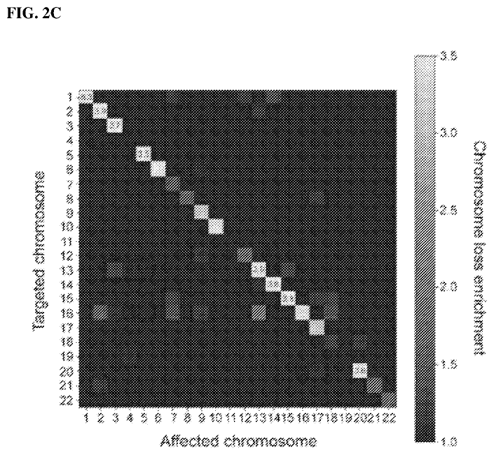

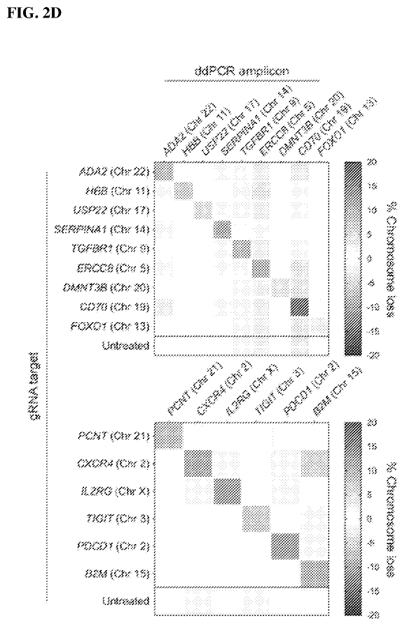

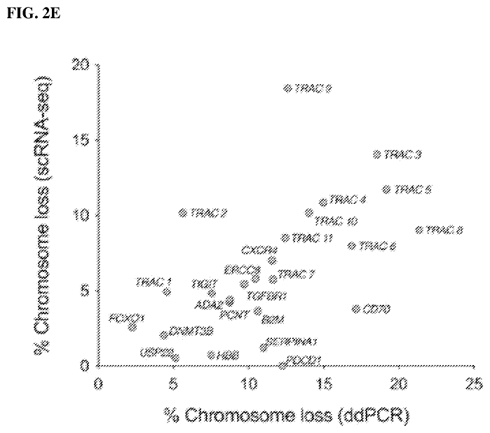

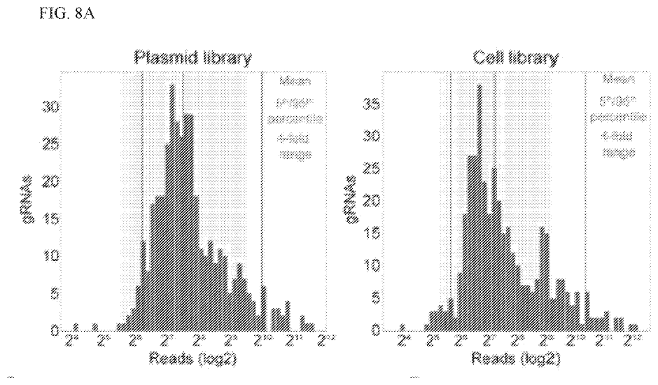

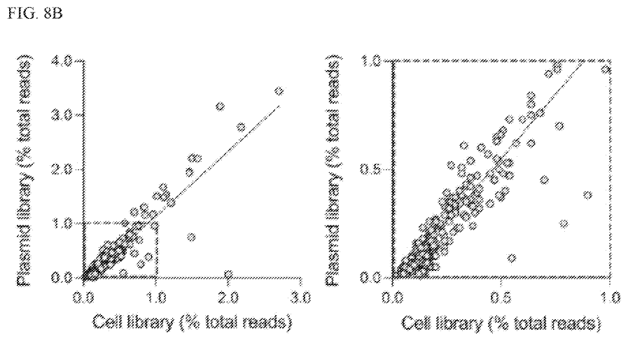

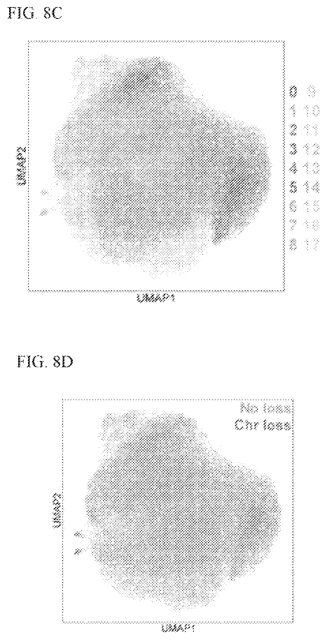

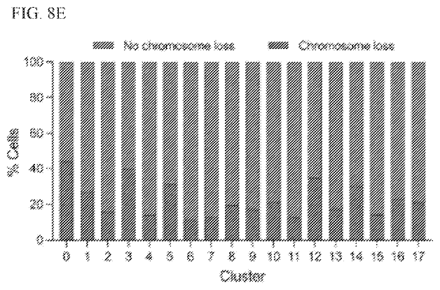

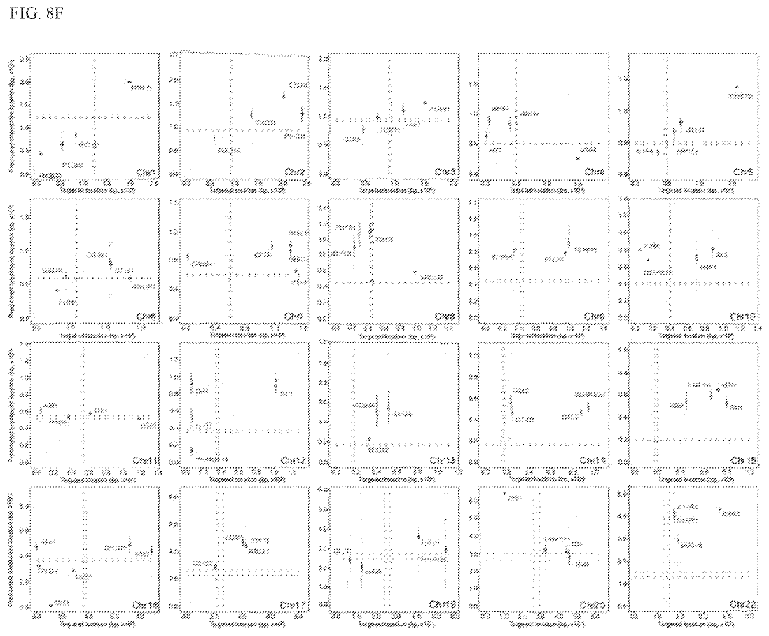

To determine the generality of chromosome loss after genome editing, a comprehensive CRISPR screen was conducted, using a library of 384 unique gRNAs targeting 3-7 genes on every somatic chromosome (92 genes in total) with four unique gRNA sequences targeting each gene (FIGS. 2A, 8A, and 8B). CROP-seq was used to track gRNAs delivered to individual cells because it avoids the template switching observed with other methods^{19,20} and because it has been successfully deployed in primary human T cells.²¹ Targets relevant to T cell genome engineering were prioritized, such as TRAC,^{1,22,23} TRBC,¹ PDCD1,¹ B2M,²⁴ IL2RA,²³ CXCR4,²⁵ and CIITA,²⁶ as well as other targets of interest for clinical genome editing such as BCL11A^{27,28} and HBB^{29,30} for the treatment of sickle cell disease, TTR to treat transthyretin amyloidosis,³¹ HTT to treat Huntington's disease,³² and SERPINA1 to treat alpha-1-antitrypsin deficiency.³³

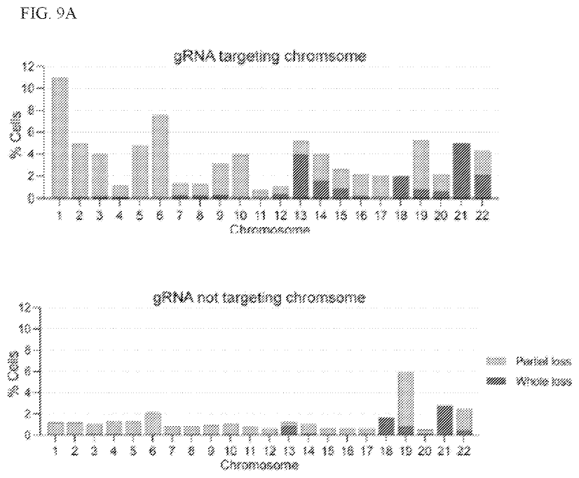

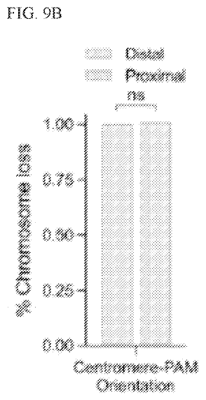

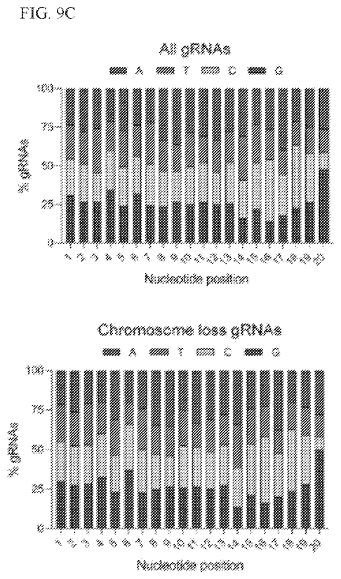

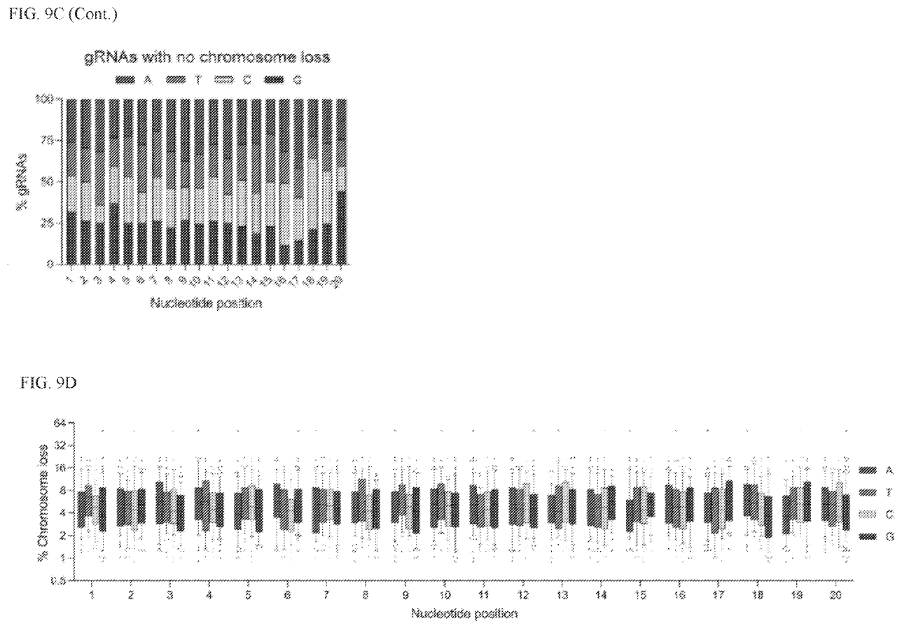

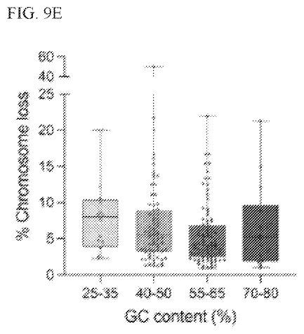

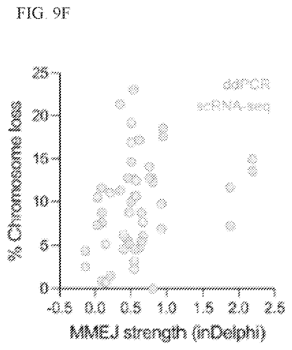

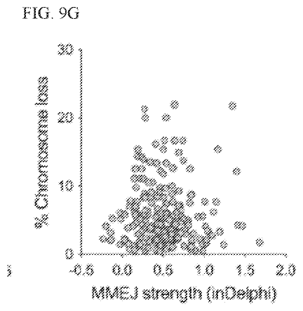

Using a previously established computational pipeline on the CROP-seq dataset, the breakpoints and gene dosage were determined for 92 different genes targeted by Cas9 (FIGS. 7C, 7D, 7E, and 7F). For numerous genes targeted in the screen, significant enrichment was observed for loss of the targeted chromosome in cells with a corresponding gRNA compared to cells with a gRNA targeting a different chromosome (FIG. 2B). Chromosome loss above background levels occurred with 55% (201/364) of all gRNAs, for 89% (82/92) of all genes targeted, and in 100% (22/22) of all chromosomes targeted. Across all gRNAs, 3.25% of targeted cells had detectable whole or partial chromosome loss (FIG. 9A). For cells with a non-targeting gRNA, no detectable chromosome loss was identified on any somatic chromosome. Enrichment for chromosome loss was much higher in chromosomes targeted by Cas9 compared to non-targeted chromosomes (FIG. 2C), suggesting that this phenomenon is an outcome of target-specific cleavage during Cas9-mediated genome editing.

This genome-scale chromosome loss was validated by selecting 15 gRNAs from the library and individually nucleofecting them as Cas9 RNPs into T cells. ddPCR was used to measure chromosome loss at various sites in the genome and showed greater levels of chromosome loss at the targeted site compared to non-targeted sites for nearly all gRNAs (FIG. 2D). Additionally, rates of chromosome loss were highly correlated (Spearman's correlation=0.59) with those estimated by scRNA-seq (FIG. 2E).

Further analyses of the CROP-seq screen to investigate the contribution of the Cas9 gRNA sequence revealed no influence by gRNA binding orientation (FIG. 9B), nucleotide sequence (FIGS. 9C and 9D), or GC content on chromosome loss (FIG. 9E). The computationally predicted dominant end-joining repair pathway, namely non-homologous end joining (NHEJ) or microhomology-mediated end joining (MMEJ), for each gRNA target also did not show a strong correlation with chromosome loss (FIGS. 9F and 9G). However, a moderate correlation was observed between the distance of each targeted gene from the centromere and the rate of chromosome loss induced (FIG. 9H), with gRNAs targeting closer to the centromere showing higher levels of chromosome loss.

### Chromosome Loss Accompanies Transcriptional Signatures of DNA Damage Response, Apoptosis, and Quiescence

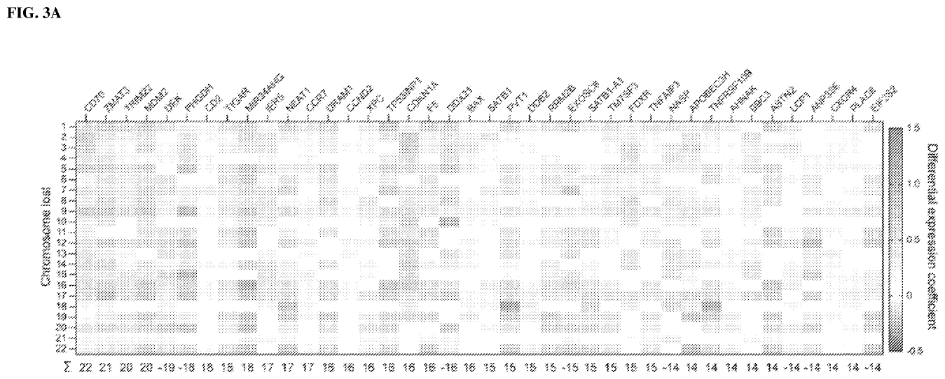

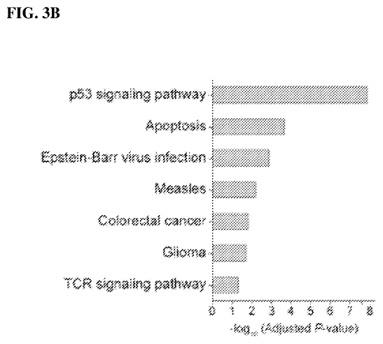

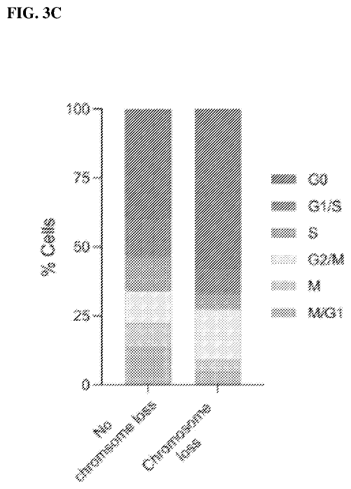

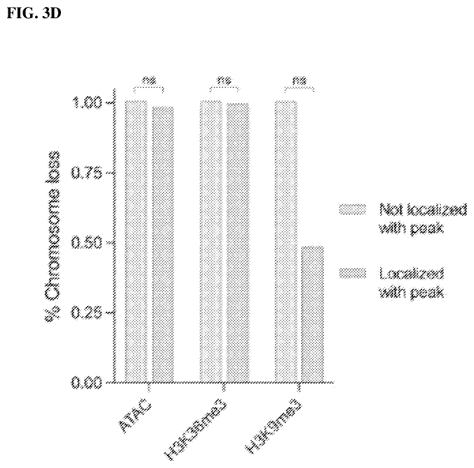

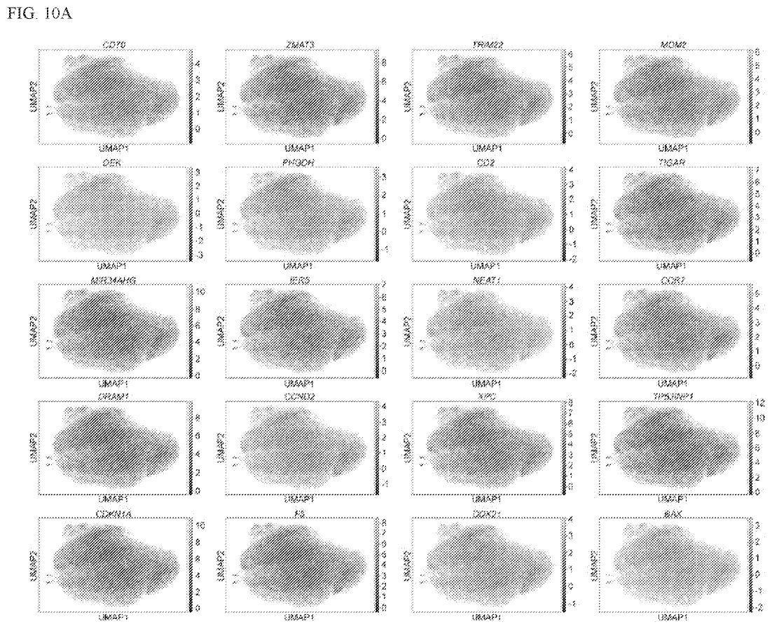

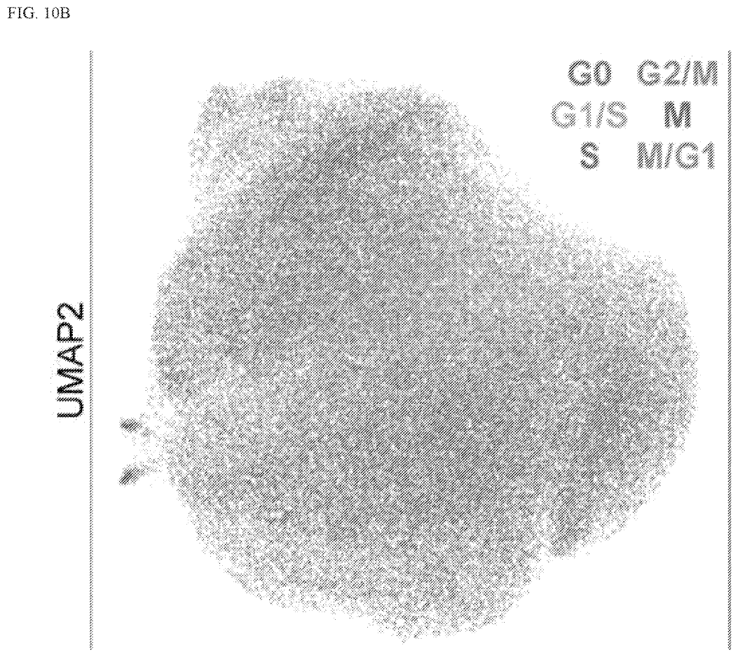

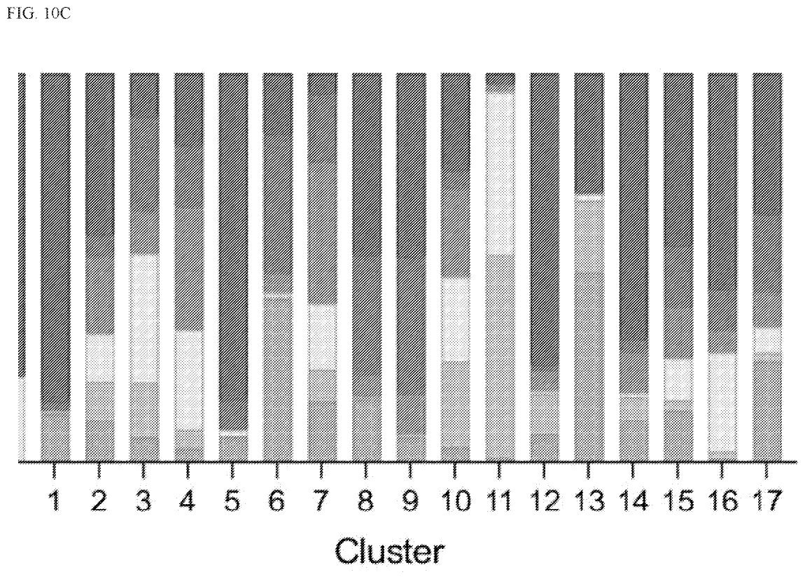

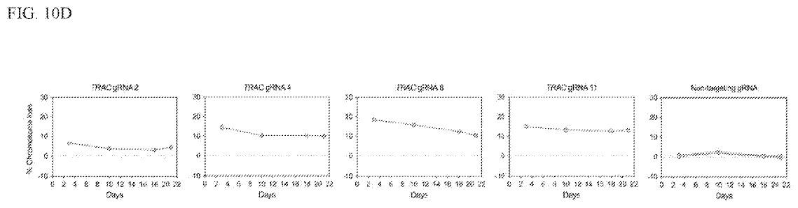

To assess the functional effects of Cas9-induced chromosome loss, differential gene expression analysis between Cas9-edited T cells with or without chromosome loss was performed. Genes that were differentially expressed across groups of cells with different chromosomes lost were identified (FIGS. 3A and 10A). CD70, for example, was significantly upregulated in every group of cells with chromosome loss regardless of which chromosome was lost, and MDM2 was significantly upregulated in every group of cells with chromosome loss except those that lost chromosomes 13 or 18. Meanwhile, PHGDH was downregulated in every group of cells with chromosome loss except those that lost chromosomes 6, 10, 18, or 21. The numerous genes that were differentially expressed across cells with various chromosomes lost suggests that these findings are not a result of expression changes from lowered dosage of the target gene, but a general influence of Cas9-induced chromosome loss.

Previous studies have demonstrated that a single Cas9-induced DSB can lead to p53 upregulation.³⁴ Consistent with this finding, gene ontology analysis revealed the p53 DNA damage response and general apoptosis pathways were the most significantly overexpressed gene modules in cells with Cas9-induced chromosome loss (FIG. 3B). An increase in cell cycle markers associated with the G0 phase, and a decrease in those associated with the S phase for T cells with chromosome loss compared to those without, was observed (FIGS. 3C, 10B, and 10C), suggesting p53-induced cell cycle arrest. The results of both the differential gene expression analysis and cell cycle analysis indicate reduced fitness in T cells with Cas9-induced chromosome loss.

The relationship between epigenetic markers and chromatin accessibility with Cas9-induced chromosome loss in primary human T cells was investigated; however, no significant correlation was found between these epigenetic factors and chromosome loss (FIG. 3D).

T Cells with Chromosome Loss Persist Ex Vivo but with Reduced Fitness and Proliferation

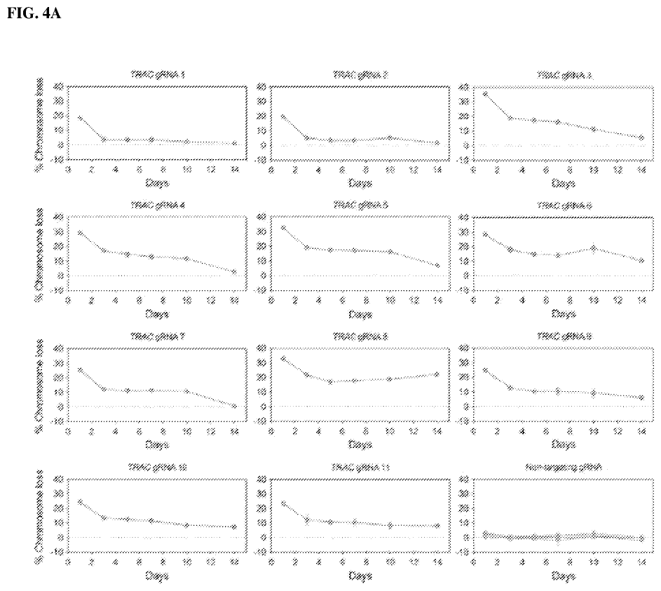

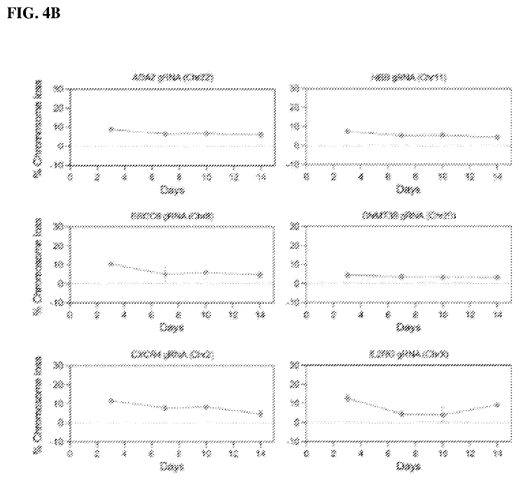

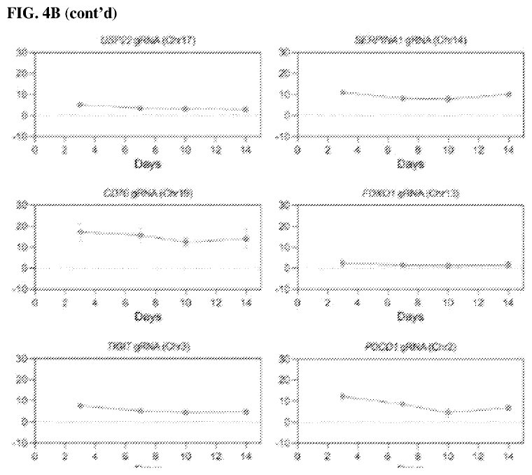

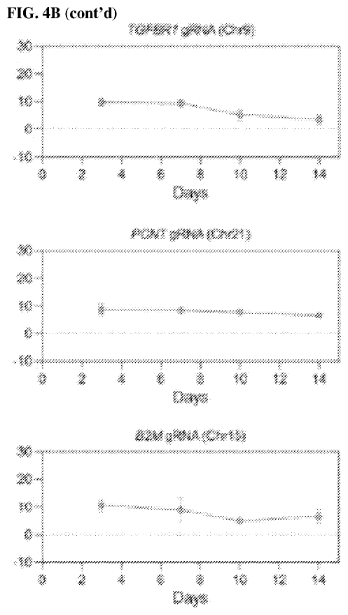

To determine whether T cells with Cas9-induced chromosome loss persist over time, ddPCR was used to measure the extent of chromosome loss at various timepoints during ex vivo culture. Timepoints over 2-3 weeks, which is a similar length of ex vivo culture compared to the manufacturing protocols of clinical trials with Cas9-edited T cells, were chosen.^{1,7} As expected, T cells treated with Cas9 RNP targeting TRAC showed the highest levels of DSBs one day after nucleofection (FIG. 4A), when Cas9 RNP is still present within cells and DNA repair is ongoing.^{35,36} By day three post-treatment, DSBs plateaued until day 14 with most conditions showing a slight downward trend (FIG. 4A). Since Cas9 RNP-mediated cleavage and DNA repair go to completion within 24 hours,³⁷ it was posited that DSBs measured at day three and beyond are from unrepaired DSBs which were considered chromosome loss.

The temporal dynamics of chromosome loss over a longer period was evaluated by repeating the experiment over the course of 21 days using four of the 11 TRAC-targeting gRNAs. Again, levels of chromosome loss showed a slight reduction over the three weeks, with detectable chromosome loss at the last timepoint remaining above that of non-targeting controls (FIG. 10D).

To test the possibility that targeting a specific gene or chromosome may affect chromosome loss or T cell viability, the Cas9 nucleofection and genomic DNA ddPCR were repeated with 15 gRNAs targeting other genes throughout the genome. Culturing the genome edited T cells for 14 days and measuring chromosome loss at various timepoints throughout, a gradual reduction in chromosome loss over time was observed (FIG. 4B). These findings show that chromosome loss in T cells is observable as far out as 2-3 weeks, across multiple targeted genes and chromosomes. However, the gradual decrease in chromosome loss over time suggests a fitness disadvantage for T cells with this genomic aberration.

It was tested whether chromosome loss was associated with a proliferative disadvantage in T cells. TRAC-edited T cells were stained with a cell proliferation dye and cultured for five days. Cells that underwent the highest and lowest amounts of proliferation, based on dye intensity, were sorted and chromosome loss was measured between the two groups. Chromosome loss in the highest proliferating quartile was identifiable but statistically lower than chromosome loss in the lowest proliferating quartile (FIG. 4C), which suggests that Cas9-induced chromosome loss confers a proliferative disadvantage.

### Gene Insertion Via Homology-Directed Repair Results in Chromosome Loss

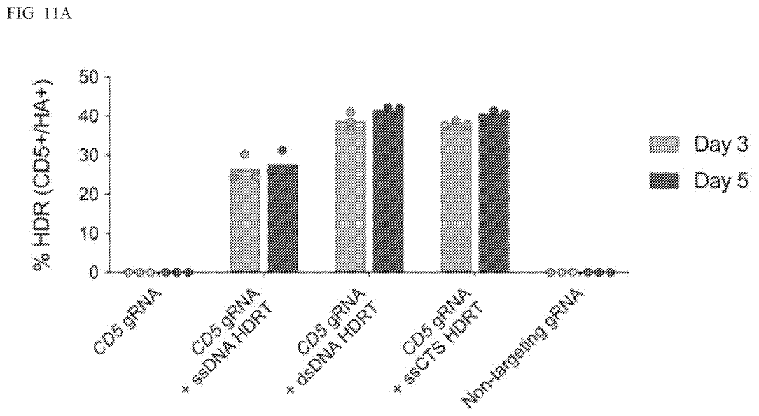

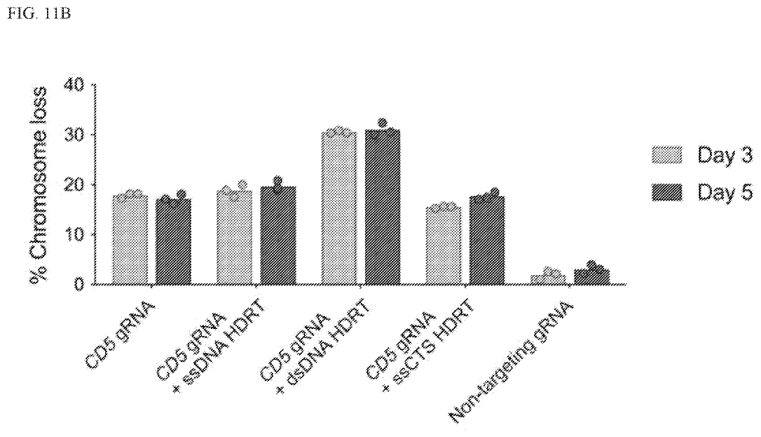

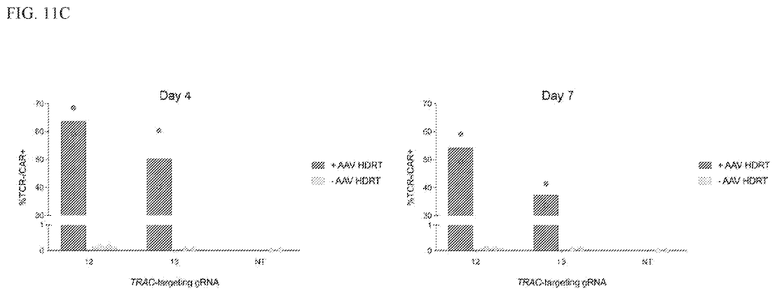

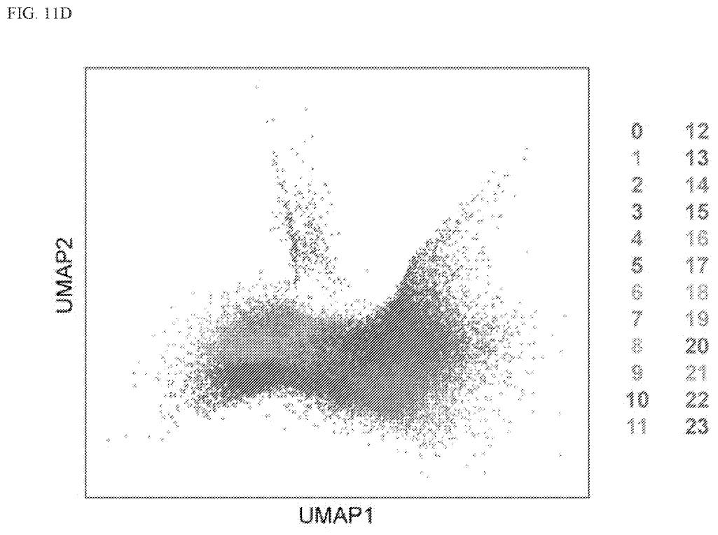

Targeted chromosome loss can occur when using Cas9 to disrupt a desired gene, which predominantly occurs through end-joining DNA repair pathways. Tremendous effort has also been invested toward using Cas9 genome editing to correct a mutation or insert a gene by homology-directed repair (HDR). Since end-joining repair and HDR are divergent DNA repair pathways that involve different proteins, undergo different amounts of resection of the DSB ends, and occur in different stages of the cell cycle,³⁸ it was asked whether chromosome loss after Cas9-mediated genome editing also occurs during HDR. To explore this, Cas9 RNPs with a gRNA targeting CD5 and various oligonucleotide HDR templates to integrate a short hemagglutinin (HA) tag in-frame with CD5 were used.³⁹ Successful generation of CD5+/HA+ cells via HDR peaked as high as ~40% three or five after nucleofection (FIG. 11A). DdPCR was performed to quantify chromosome loss rates at both timepoints and observed similar levels of chromosome loss in CD5-targeted cells with an HDR template compared to CD5-targeted cells without an HDR template (FIG. 11B). Additionally, using CD5 and other T cell surface markers, fluorescence-activated cell sorting was used to enrich for cells without chromosome loss; however, we observed no reduction in chromosome loss (Supplementary Note 1).

### Pre-Clinical CAR T Cell Generation Results in Chromosome Loss

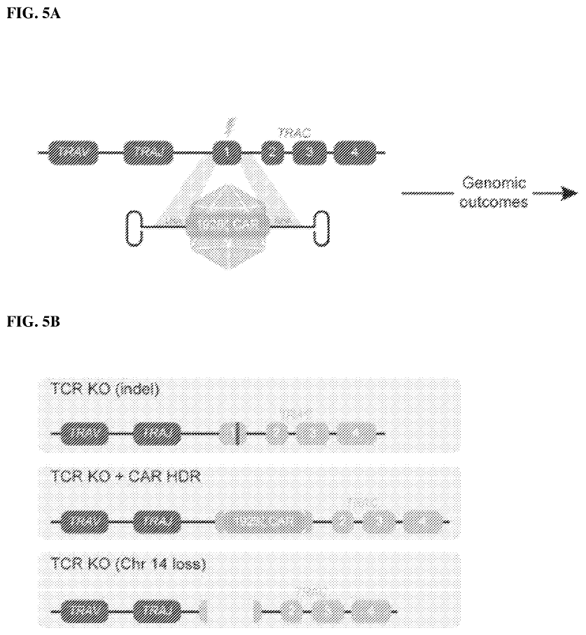

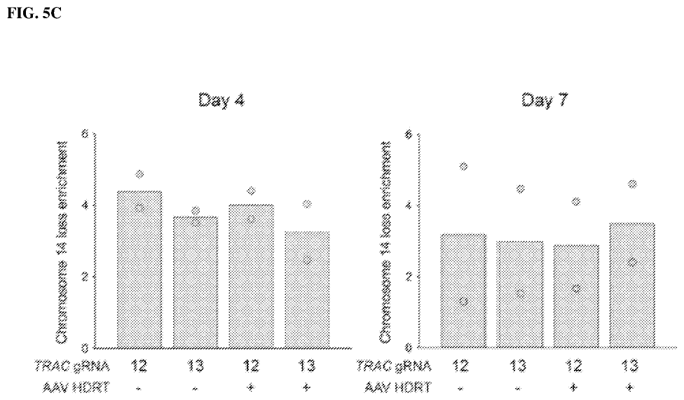

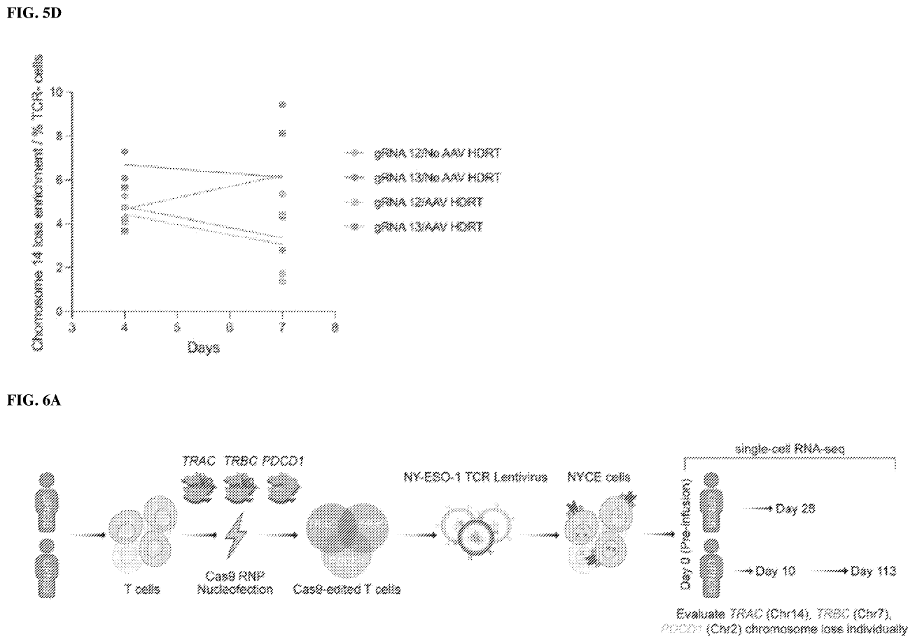

While CAR T cells and transgenic TCR T cells are currently manufactured using a retrovirus or lentivirus to semi-randomly integrate the retargeting transgene, researchers have also used Cas9 and HDR to precisely insert the transgene within the TRAC locus.^{22,39,40} This approach, which utilizes the native TRAC promoter to control expression of the CAR or retargeted TCR, offers advantages including uniform expression, minimal tonic signaling, and simultaneous disruption of the endogenous TCR. To explore whether chromosome loss occurs when generating pre-clinical CAR T cells via HDR, primary human T cells were nucleofected with Cas9 complexed with one of two TRAC-targeting gRNAs or a non-targeting gRNA. Just after nucleofection, recombinant adeno-associated virus 6 (AAV6) encoding a 1928ζ CAR as an HDR template was added to the T cells (FIGS. 5A and 5B).^{41,42} Both the reduction of TCR expression and the gain of CAR expression were observed in two independent nucleofections; cells from one nucleofection were subjected to scRNA-seq at day four post-manufacturing, while cells from the other were subjected to scRNA-seq at day seven post-manufacturing. Overall rates of CAR integration via HDR were ~34-69% (FIG. 11C). In all conditions that received a TRAC-targeting gRNA, regardless of day or whether an HDR template was present, an enrichment in chromosome 14 loss, compared to conditions with a non-targeting gRNA, was observed (FIGS. 5C, 11D, and 11E). When chromosome 14 loss enrichment was normalized to editing efficacy, since day four and day seven CAR T cells were engineered independently, a slight decrease in chromosome 14 loss enrichment over time was observed (FIG. 5D). Together, these data suggest that chromosome loss is a general phenomenon that occurs in Cas9-edited T cells, regardless of the DNA repair pathway involved. These findings also indicate that cells with chromosome loss are present among pre-clinical, Cas9-edited CAR T cells, highlighting the importance of understanding and mitigating this genotoxicity in the context of engineered T cell therapies.

Investigation of Chromosome Loss in Cas9-Edited T Cells from Clinical Trial Patients

Studies thus far have focused on ex vivo culturing of T cells; it is not yet known how these findings translate in vivo. A first-in-human phase 1 clinical trial (clinicaltrials.gov, trial NCT03399448) was conducted, in which Cas9 genome edited T cells were administered to patients with advanced, refractory cancer.¹ Autologous T cells from three cancer patients were collected and electroporated with three different Cas9 RNPs, simultaneously targeting TRAC, TRBC, and PDCD1. These edited T cells were then transduced with a lentivirus encoding an HLA-A2*0201-restricted TCR specific to a peptide from the NY-ESO1 and LAGE-1 cancer antigens, resulting in engineered T cells (NYCE). NYCE cells were infused back into the patients and were found to be well-tolerated.

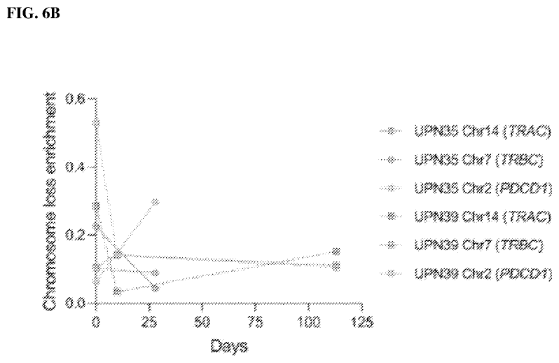

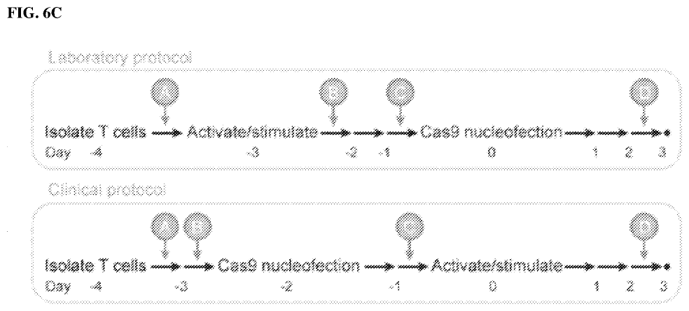

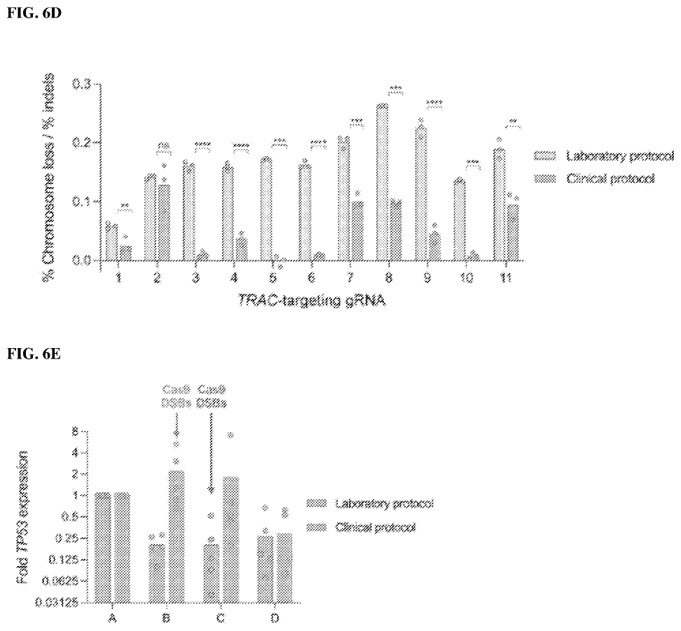

To investigate whether clinical manufacturing of a Cas9-edited adoptive T cell therapy results in levels of chromosome loss similar to those observed in the laboratory studies, scRNA-seq data from NYCE cells of two patients at various timepoints throughout the clinical trial were analyzed. Cells were collected from patient UPN35 prior to infusion and at day 28 post-infusion, while cells from patient UPN39 were collected prior to infusion as well as at days 10 and 113 post-infusion (FIG. 6A). Similar to the laboratory experiments, gene dosage was inferred at each of the target chromosomes (TRAC, Chr14; TRBC, Chr7; PDCD1, Chr2) and partial and whole chromosome loss was looked for. Surprisingly, extremely low levels of chromosome loss at the targeted chromosomes was observed, which were not enriched compared to background levels at non-targeted chromosomes (FIG. 6B).

### Order of Operations During Cas9 Genome Editing Impacts Chromosome Loss

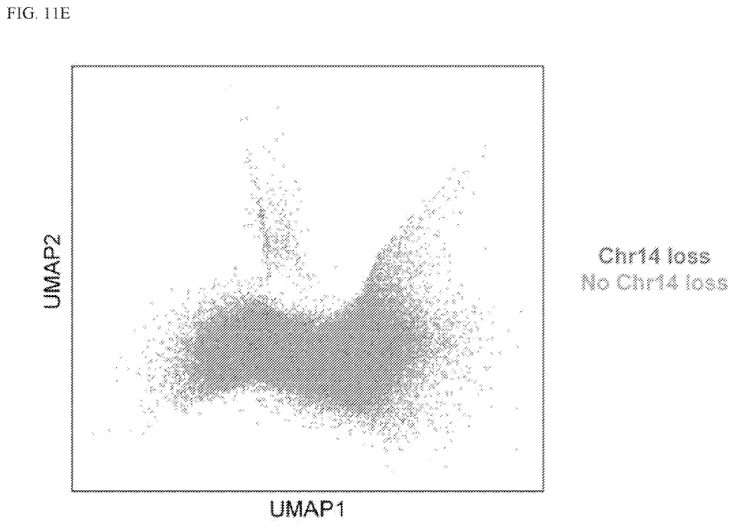

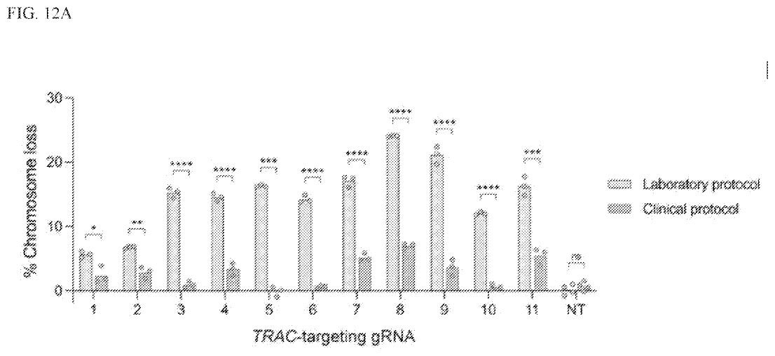

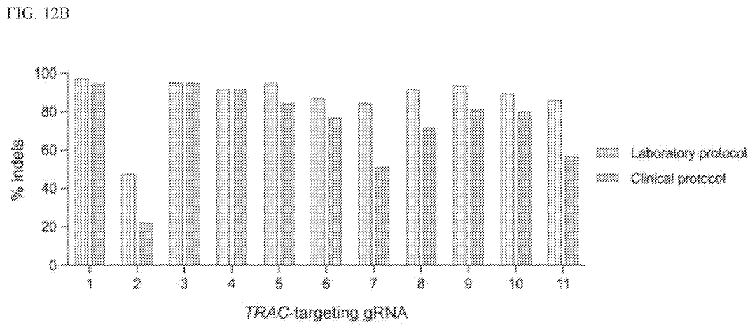

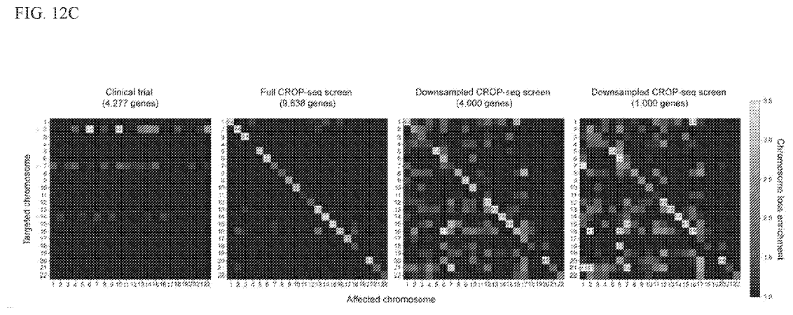

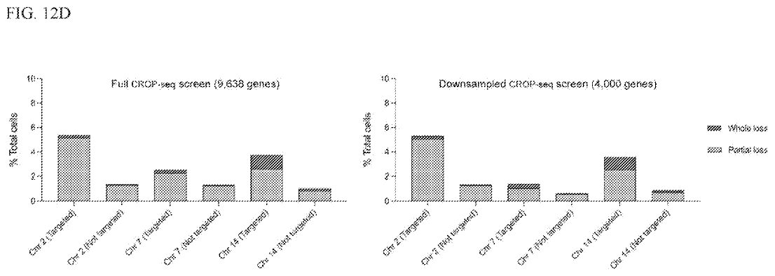

It was asked whether the discrepancy between the high rates of chromosome loss in the laboratory studies versus the low rates in the clinical trial could be attributed to the engineered T cell manufacturing protocol. In laboratory studies, T cells were activated and stimulated prior to introducing Cas9 RNP and generating DSBs, while in clinical trial, Cas9 RNP was introduced and DSBs were generated prior to activating and stimulating the T cells (FIG. 6C). Cas9 genome editing of TRAC was performed in primary human T cells using the laboratory protocol (activation/stimulation followed by DSBs) and simulating the clinical protocol (DSBs followed by activation/stimulation) in parallel. Across all 11 TRAC-targeting gRNAs, markedly lower levels of chromosome loss were observed using the clinical protocol compared to the laboratory protocol (FIG. 12A). However, genome editing with the clinical protocol on average resulted in ~14% lower indels compared to the laboratory protocol (FIG. 12B). To control for this difference, the rate of chromosome loss was normalized to the rate of indels generated per gRNA; a statistically significant reduction in chromosome loss was still observed with the clinical protocol as compared to the laboratory protocol with 10 out of 11 gRNAs (FIG. 6D).

Previous studies have shown that p53, a key protein in cell cycle regulation and apoptosis, also regulates T cell activation. Downregulation of p53 is critical for murine T cell proliferation.⁴³ Additionally, p53-mediated apoptosis has been shown as a mechanism for selecting against aneuploid cells.⁴⁴ Therefore, it was tested whether differences in manufacturing protocol influenced p53 levels, and how that related to the chromosome loss that was observed. Expression of TP53, which encodes p53, was measured via RT-qPCR at different timepoints throughout both T cell genome editing protocols. Similar to what was observed in murine T cells, expression of p53 was lowered in both protocols after human T cell activation/stimulation (FIG. 6E). The mean TP53 expression across five biological donors was >10 times higher immediately prior to Cas9-induced DSBs in the clinical protocol compared to the laboratory protocol (FIG. 6E). Thus, TP53 expression during Cas9-induced DSBs inversely correlated with rates of chromosome loss in T cells between the two protocols. For the clinical protocol, the higher expression of this key DNA damage response factor during Cas9-induced DSBs could select against cells with chromosome loss and explain the dramatically lower rates that were observed. Implementation of this modified protocol for Cas9 genome editing in T cells represents a simple adjustment that could substantially mitigate chromosome loss in future research and clinical studies.

### FIGURES

FIG. 1A-1H. CRISPR-Cas9 genome editing of TRAC results in whole and partial chromosome loss. (A) Cas9 gRNA target sites tiled across the first exon of TRAC on chromosome 14. (B) Gene dosage from transcriptome-wide scRNA-seq of T cells treated with Cas9 and a non-targeting gRNA (top heatmap) or TRAC-targeting gRNA (bottom heatmap). Each individual row corresponds to a single cell and each column corresponds to a specific gene and its genomic position, grouped by chromosome (outlined in black). Red represents increase in gene dosage while blue represents decrease in gene dosage. Rows outlined in black represent cells treated with different TRAC-targeting gRNAs. Blue arrows highlight chromosome 14, where TRAC is located. (C) Distribution of computationally predicted chromosome 14 breakpoints in cells predicted to have a chromosomal loss event. The distribution is an aggregate of 11 different TRAC-targeting gRNAs (all within ~300 bp) in cells with partial chromosome loss. (D) Representative single cell chromosome 14 gene dosage plots illustrating a cell with no chromosome loss (left), whole chromosome loss (middle), or partial chromosome loss (right). Gene dosage was normalized to non-targeting samples. Gray shaded area (gene dosage of 0.95-1.05) represents normal gene dosage (2 copies). Blue shaded area (gene dosage of <0.95) represents reduction in gene dosage (1 copy). Dotted lines represent the centromere, black lines represent the Cas9 target site, and the red line represents the computationally predicted breakpoint. (E) Quantification of whole and partial chromosome 14 loss across all gRNAs from scRNA-seq. NT indicates non-targeting gRNA. (F) Schematic of ddPCR assay to measure chromosome loss. The yellow lightning bolt represents the Cas9 target site. The detection of both HEX and FAM probes indicates no DSB or repaired DSB (top illustration). The detection of the HEX probe but not the FAM probe indicates an unresolved DSB that represents chromosome loss (bottom illustration). (G) Quantification of chromosome loss at the Cas9 target site across all gRNAs from ddPCR (n=3, n=2 biological donors). Numbers next to each point represent the TRAC-targeting gRNA. NT indicates non-targeting gRNA and represents samples from four different ddPCR amplicons. Error bars represent the standard deviation from the mean. Dashed line represents linear regression line of best fit and shaded region represent 95% confidence intervals (Slope=1.082, R²=0.9853). (H) Comparison of chromosome 14 loss between TRAC-targeting conditions where the PAM is distal (Centromere-PAM distal) or proximal (Centromere-PAM proximal) to the centromere relative to the gRNA spacer sequence. Chromosome 14 loss was measured by scRNA-seq (n=1 biological donor) or ddPCR (n=3, n=2 biological donors). P-values are from Welch's unpaired t-tests and are from left to right 0.8689 and 0.7338. ns=not significant.

FIG. 2A-2E: Genome-scale CRISPR-Cas9 screen reveals target-specific chromosome loss. (A) Workflow for a CRISPR-Cas9 screen to estimate chromosome loss in T cells. Primary human T cells were transduced with a CROP-seq lentiviral library expressing one of 384 gRNAs. Cells were then nucleofected with Cas9 protein, before GFP+ cells (co-expressed on the CROP-seq gRNA vector) were enriched via fluorescence-activated cell sorting. Enriched cells were subject to scRNA-seq and downstream analysis. (B) Quantification of targeted chromosome loss enrichment for each target gene. Each of the 92 bars represents the combination of four unique gRNAs targeting the same gene. Chromosome loss enrichment was calculated relative to the baseline loss per chromosome in cells containing a gRNA targeting a different chromosome. Error bars represent 95% confidence intervals. (C) Chromosomal loss enrichment at each somatic chromosome across all gRNAs. Rows represent the chromosome targeted by the Cas9 gRNA. Columns represent the chromosome analyzed for chromosome loss. (D) Chromosome loss measured by ddPCR at 15 different Cas9 target sites across the genome. Rows titles indicate the identity of the gRNA used. Column titles indicate the site in the genome that was analyzed via ddPCR. Heatmap values represent the mean of replicates (n=3, except n=2 for B2M target column). (E) Correlation between chromosome loss from 25 gRNAs as measured by scRNA-seq and ddPCR. Spearman's correlation=0.59, **P=0.0017 (two-tailed).

FIG. 3A-3D. Genetic and epigenetic factors influence Cas9-induced chromosome loss. (A) Heatmap of differentially expressed genes in cells with chromosome loss compared to cells without chromosome loss. Cells with chromosome loss were divided into 22 groups depending on which somatic chromosome was lost (rows), and differentially expressed genes were individually investigated (columns). Upregulated genes are shown in red while downregulated genes are shown in blue. Genes were given a score of 1 (upregulated), −1 (downregulated), or 0 (no difference) for each chromosome loss group. Summed gene scores across all chromosome loss groups is shown below; genes with a score >|13| are displayed. (B) Gene ontology analysis based on differential gene expression. The most significantly upregulated modules are displayed. (C) Cell cycle analysis based on expression profiles. The percentage of cells in each cell cycle phase were quantified for cells with no chromosome loss or cells with chromosome loss. (D) Influence of epigenetic marks on chromosome loss. The gRNA sequence for cells with or without chromosome loss was analyzed for localization within ±75 bp of an epigenetic marker peak. P-values were calculated using a two-sided Fisher's Exact Test and are from left to right (ATAC) 0.365496, (H3K36me3) 0.789824, and (H3K9me3) 0.305706. ns=not significant.

FIG. 4A-4C. Cas9-induced chromosome loss persists for weeks but results in reduced fitness and proliferation. (A) ddPCR measurements of chromosome loss at the Cas9 TRAC target site over 14 days. Error bars represent the standard deviation from the mean (n=3). Day 3 results were additional used as the donor 2 (female) results shown in FIG. 1g. (B) ddPCR measurements of chromosome loss for 15 different gRNAs targeted to sites across the genome over 14 days. Error bars represent the standard deviation from the mean (n=3, except n=2 for B2M). Day 3 results were additionally used for the diagonal values in the heatmaps of FIG. 2d. (C) Measurement of chromosome loss across T cells of varying proliferative capacity. T cells were stained with CellTrace Violet (CTV) and cultured for five days before sorting the top and bottom quartile (top panel). ddPCR was used to measure chromosome loss in lowly proliferative (CTV high) and highly proliferative (CTV low) populations (bottom panel). NT=non-targeting gRNA. Non-targeted samples evaluated for chromosome loss at the gRNA 2 or gRNA 8 amplicon were combined into a single column (n=3 for each of the two different ddPCR amplicons). P-values are from Welch's unpaired t-tests and from left to right are 0.002970, 0.002970, and 0.275572.

FIG. 5A-5D. Pre-clinical CAR T cell production via homology-directed repair results in chromosome loss. (A) Strategy to generate CAR T cells via HDR with Cas9. AAV6 encoding a 1928ζ CAR between left and right homology arms (LHA and RHA, respectively) serves as a template for HDR after Cas9 cleavage (yellow lightning bolt) of TRAC. (B) Three potential genomic outcomes after Cas9 HDR: indels that disrupt TCR expression (top), insertion of the CAR transgene that simultaneously disrupts TCR expression (middle), and chromosome loss that disrupts TCR expression (bottom). (C) Quantification of chromosome 14 loss enrichment across two TRAC-targeting gRNAs with or without an AAV HDR template from scRNA-seq (n=2 biological donors). Two separate batches of CAR T cells were manufactured, before being subjected to scRNA-seq four or seven days after generation. Chromosome 14 loss enrichment was calculated relative to T cells treated with Cas9 and a non-targeting gRNA. (D) Chromosome 14 loss enrichment over time, normalized to Cas9 editing efficacy (n=2 biological donors). Editing efficacy was determined by the percentage of TCR negative cells as measured via flow cytometry.

FIG. 6A-6E. Clinical CRISPR-Cas9 genome editing protocol in patient T cells mitigates chromosome loss. (A) Strategy to investigate chromosome loss in two clinical trial patients with CRISPR-edited T cells. Two patients with refractory cancer had T cells isolated, nucleofected with TRAC, TRBC, and PDCD1-targeting Cas9 RNPs, and transduced with a lentivirus encoding an NY-ESO-1 TCR (NYCE cells). Cells were subjected to scRNA-seq prior to infusion (Day 0) and as well as at different time points post-infusion (Days 10, 28, and/or 113). (B) Chromosome loss enrichment on chromosome 14 (TRAC), chromosome 7 (TRBC), or chromosome 2 (PDCD1) at different timepoints for both patients. Enrichment was calculated relative to non-targeted chromosomes (all chromosomes but 2, 7, and 14). Day 0 represents NYCE cells prior to infusion, while other later timepoints represent NYCE cells that were collected after circulation in vivo. (C) Diagram of two different protocols for Cas9 genome editing of primary human T cells. The laboratory protocol (top) consisted of activating/stimulating cells prior to Cas9 nucleofection, and was used throughout this study. The clinical protocol (bottom) consisted of nucleofecting cells with Cas9 prior to activating/stimulating and is representative of our clinical trial. (D) Relative chromosome loss with 11 different TRAC-targeting gRNAs using the laboratory or clinical protocol in primary human T cells. Chromosome loss was normalized to the indel efficacy. P-values are from Welch's unpaired t-tests and from left to right are 0.008320, 0.111695, 0.000052, 0.000076, 0.000159, 0.000073, 0.000125, 0.000087, 0.000073, 0.000050, and 0.001416. (E) Fold TP53 mRNA expression during the laboratory or clinical protocols for Cas9 genome editing of primary human T cells (n=5 biological donors). Data points are the mean of two technical replicates. X-axis letters correspond to timepoints in FIG. 6c. “Cas9 DSBs” represents the timepoints in the laboratory or clinical protocols where Cas9 was nucleofected into T cells to generate DSBs.

FIG. 7A-7I. CRISPR-Cas9 genome editing results in chromosome loss regardless of guide RNA orientation or genomic position. (A) TCR disruption from Cas9 genome editing of TRAC. TCR expression was measured via flow cytometry at 3, 7, and 10 days post-nucleofection. NT indicates non-targeting gRNA. (B) Indels at the TRAC locus (targeting) from Cas9 genome editing as measured by next-generation sequencing. Cells treated with a non-targeting gRNA were evaluated for indels at each of the TRAC target sequences (non-targeting). (C) UMAP projection of T cells edited with a TRAC-targeting or non-targeting gRNA. (D) The UMAP projection of T cells within the TRAC editing experiment was overlayed with estimations of whether the cell had chromosome 14 loss or not (whole or partial chromosome loss). (E) Percentage of cells with chromosome 14 loss per cluster (see FIG. 7D). (F) Quantification of chromosome 14 loss enrichment across 11 different TRAC-targeting gRNAs. Chromosome 14 loss enrichment was calculated relative to T cells treated with Cas9 and a non-targeting gRNA. (G) Schematic of gRNA orientation relative to the centromere. Cas9 targets where the PAM was proximal to the centromere (red) relative to the target DNA sequence were considered centromere-PAM proximal (blue), while Cas9 targets where the PAM was distal to the centromere relative to the target DNA sequence were considered centromere-PAM distal (green). (H) Comparison of TCR disruption (by flow cytometry or next-generation sequencing) and chromosome 14 loss as measured by scRNA-seq (top) or ddPCR (bottom). (I) Chromosome 14 loss by scRNA-seq (combination of whole and partial chromosome 14 loss, top) or ddPCR (mean of n=3 replicates, n=2 biological donors, bottom) for each gRNA based on target position within the TRAC gene. Gray line indicates the first exon of TRAC (274 bp).

FIG. 8A-8F. CROP-seq reveals genome-scale breakpoints and chromosome loss. (A) Distribution of next-generation sequencing reads for each gRNA in the CROP-seq gRNA library as a plasmid (left) or once integrated into T cells via lentivirus (right). The mean is shown as a red line, 5^{th} and 95^{th} quartiles are shown as green lines, and a 4-fold range to either side of the mean is shown as a pink shaded area. (B) Correlation between gRNA reads in the plasmid library and cell library. A zoomed in perspective (blue dashed line, left) is shown in a separate panel (blue dashed line, right). Dashed gray line represents the linear regression line of best fit (Slope=1.189, R²=0.8348). (C) UMAP projection of T cells from the CROP-seq screen. (D) The UMAP projection of T cells within the CROP-seq screen was overlayed with estimations of whether the cell had a targeted chromosome loss or not (whole or partial chromosome loss). (E) Percentage of cells with targeted chromosome loss per cluster (see FIG. 8C). (F) Predicted breakpoint location versus intended gRNA target location from the CROP-seq screen. gRNAs are grouped by targeted chromosome. Chromosomes 18 and 21 are omitted because no partial chromosome loss was detected. Red data points represent the mean and 95% confidence interval.

FIG. 9A-9H. Influence of genetic context and Cas9 gRNA sequence on chromosome loss. (A) Partial and whole chromosome loss from the CROP-seq screen. Chromosome loss was quantified at chromosomes where the gRNA was targeting that specific chromosome (left) or at chromosomes not targeted by the gRNA (right). Chromosome loss at chromosomes not targeted by the gRNA was used to calculate baseline noise. (B) Influence of gRNA orientation on chromosome loss. gRNAs where the PAM is distal to the centromere relative to the gRNA target sequence were compared against gRNAs where the PAM is proximal to the centromere relative to the gRNA target sequence. P-value was calculated using a two-sided Fisher's Exact Test and was 0.592413. ns=not significant. (C) Distribution of nucleotides across each position of the gRNA spacer within the CROP-seq library. Distribution for all gRNAs (left), gRNAs that resulted in chromosome loss (middle), and gRNAs that did not result in chromosome loss (right). (D) Chromosome loss by nucleotide identity across each position of the gRNA spacer within the library. (E) Chromosome loss by gRNA spacer sequence GC content. gRNAs were arbitrarily binned by varying levels of GC content. (F) Chromosome loss versus computationally predicted MMEJ influence for Cas9 RNP nucleofection experiments (teal=ddPCR measurements, yellow=scRNA-seq measurements. Chromosome loss rates are identical to FIG. 2e). ddPCR Spearman's correlation=0.40, *P=0.04 (two-tailed); scRNA-seq Spearman's correlation=0.27, P=0.19 (two-tailed). (G) Chromosome loss versus computationally predicted MMEJ influence for the CROP-seq screen experiment. gRNAs with non-zero chromosome loss were plotted. Spearman correlation=−0.08, P=0.25 (two-tailed). (H) Chromosome loss by position along the target chromosome. Distance from the centromere was normalized to the length of the target chromosome. Spearman's correlation=−0.34, ***P=0.0009 (two-tailed).

FIG. 10A-10D. Cas9-induced chromosome loss is associated with differential gene expression and a fitness disadvantage. (A) UMAP projections of T cells within the CROP-seq screen. Gene expression was overlayed onto the projections for the top 20 genes that were most differentially expressed across cells that had chromosome loss. (B) UMAP projection of T cells within the CROP-seq screen overlayed with cell cycle markers (see FIG. 8C). (C) Quantification of cell cycle states across the different clusters (see FIG. 8C). (D) ddPCR measurements of chromosome loss at the Cas9 TRAC target site over 21 days. Error bars represent the standard deviation from the mean (n=3).

FIG. 11A-11E. CRISPR-Cas9 homology-directed repair results in chromosome loss. (A) HDR efficacy 3 or 5 days post-nucleofection, determined by measuring CD5+/HA+ T cells via flow cytometry. (B) Chromosome loss at the target CD5 locus via ddPCR, 3 or 5 days post-nucleofection. (C) HDR efficacy determined by measuring TCR−/CAR+ T cells via flow cytometry, four or seven days post-nucleofection. Two separate nucleofections/transductions were conducted for the different time points (n=2 biological donors). (D) UMAP projection of CAR T cells generated via Cas9 HDR. Projection is an aggregate of two biological donors and multiple time points. (E) Distribution of CAR T cells with chromosome 14 loss across the UMAP projection (whole or partial chromosome loss).

FIG. 12A-12D. Clinical CRISPR-Cas9 genome editing protocol reduces chromosome loss in T cells. (A) Chromosome loss with 11 different TRAC-targeting gRNAs or a non-targeting gRNA (NT) using the laboratory or clinical protocol in T cells (n=3). P-values are from Welch's unpaired t-tests and from left to right are 0.010220, 0.004303, 0.000063, 0.000083, 0.000170, 0.000083, 0.000063, 0.000063, 0.000063, 0.000031, 0.000224, and 0.079286. (B) Indels measured by next-generation sequencing at the TRAC locus by Cas9 genome editing with the laboratory or clinical protocol. (C) Downsampling analysis to investigate the influence of total genes on chromosome loss enrichment. Rows represent the chromosome targeted by Cas9 and its gRNA. Columns represent the chromosome analyzed for chromosome loss. Chromosomal loss enrichment for all clinical trial patients and timepoints was evaluated only when targeting chromosomes 2 (PDCD1), 7 (TRBC), and 14 (TRAC) (yellow arrows, left heatmap). 4,277 total genes were detected in the clinical trial dataset. The full CROP-seq screen dataset (9,638 genes, second from left) was downsampled to 4,000 genes (second from right) or 1,000 genes (right) to evaluate the influence of total genes on chromosomal loss enrichment. (D) Quantification of whole and partial chromosome loss at chromosomes 2, 7, and 14 from the CROP-seq screen. Chromosome loss was measured at the specific chromosome targeted by the Cas9 gRNA (targeted) or at chromosomes not targeted by the Cas9 gRNA (not targeted), from the full CROP-seq screen dataset (left) or the CROP-seq screen dataset downsampled to mimic the clinical trial dataset (right).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for modifying the genome of a mammalian cell, the method comprising contacting the mammalian cell in vitro with:
a) a gene editing composition comprising:
i) a CRISPR-Cas effector polypeptide; and
ii) a guide nucleic acid; and
b) an agent that increases the level of a p53 polypeptide in the cell,
wherein said contacting results in modification of the genome of the cell, thereby producing a modified cell.

2. The method of claim 1, wherein the agent comprises a nucleic acid comprising a nucleotide sequence encoding a p53 polypeptide.

3. The method of claim 2, wherein the nucleic acid is an mRNA.

4. The method of claim 2, wherein the nucleic acid is a recombinant expression vector.

5. The method of claim 4, wherein the recombinant expression vector is a lentiviral vector or an adeno-associated viral vector.

6. (canceled)

7. The method of claim 1, wherein the agent is a small molecule that inhibits degradation of the p53 polypeptide in the cell, thereby increasing the level of the p53 polypeptide in the cell.

8. The method of claim 7, wherein the agent is an inhibitor of MDM2.

9. The method of claim 8, wherein the agent is selected from: nutlin-1, nutlin-2, nutlin-3, RG7388, RG7775, RG7112, AMG232, CGM097, HDM201, APG115, DS-3032b, MI77301, MK-8242, APR-246, RO6839921, NVP-CGM097, JNJ-26854165, RO5353, RO2468, RO8994, HLI98, SP-141, SJ-172550, XI-006, XI-011, CTX1, RO-2443, RO-05963, MI-219, MI-888, MI-773, AM-8735, AM-8553, AM-7209, AMG-232, HDM201, MK-8242, RO6839921, DS-3032, Tenovin-1/6, Leptomycin B, Inauhzin, RITA, PK083, PK7088, ZMC1, RETRA, NSC59984, Prodigiosin, P53R3, SCH529074, CP-31398, Chetomin, and TDP665759.

10-18. (canceled)

19. The method of claim 1, wherein the gene editing composition further comprises a donor nucleic acid.

20. The method of claim 19, wherein the donor nucleic acid comprises a nucleotide sequence encoding a chimeric antigen receptor.

21. The method of claim 19, wherein the donor nucleic acid comprises a nucleotide sequence encoding a heterologous T-cell receptor.

22-28. (canceled)

29. A gene editing composition comprising:
a) a gene editing composition comprising:
i) a CRISPR-Cas effector polypeptide; and
ii) a guide nucleic acid; and
b) an agent that increases the level of a p53 polypeptide in a mammalian cell.

30. The gene editing composition of claim 29, wherein the agent comprises a nucleic acid comprising a nucleotide sequence encoding a p53 polypeptide.

31. (canceled)

32. The gene editing composition of claim 30, wherein the nucleic acid is a recombinant expression vector.

33. The gene editing composition of claim 32, wherein the recombinant expression vector is a lentiviral vector or an adeno-associated viral vector.

34. (canceled)

35. The gene editing composition of claim 29, wherein the agent is a small molecule that inhibits degradation of the p53 polypeptide in the cell, thereby increasing the level of the p53 polypeptide in the cell.

36. The gene editing composition of claim 35, wherein the agent is an inhibitor of MDM2.

37. The gene editing composition of claim 36, wherein the agent is selected from: nutlin-1, nutlin-2, nutlin-3, RG7388, RG7775, RG7112, AMG232, CGM097, HDM201, APG115, DS-3032b, MI77301, MK-8242, APR-246, RO6839921, NVP-CGM097, JNJ-26854165, RO5353, RO2468, RO8994, HLI98, SP-141, SJ-172550, XI-006, XI-011, CTX1, RO-2443, RO-05963, MI-219, MI-888, MI-773, AM-8735, AM-8553, AM-7209, AMG-232, HDM201, MK-8242, RO6839921, DS-3032, Tenovin-1/6, Leptomycin B, Inauhzin, RITA, PK083, PK7088, ZMC1, RETRA, NSC59984, Prodigiosin, P53R3, SCH529074, CP-31398, Chetomin, and TDP665759.

38. (canceled)

39. (canceled)


SEQUENCE LISTING (5 sequences)

SEQ ID NO: 1 - protein, 393 aa, Homo sapiens [sequence omitted]

SEQ ID NO: 2 - protein, 341 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 3 - protein, 346 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 4 - protein, 261 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 5 - protein, 209 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC